(19) United States
(12) Patent Application Publication
(10) Pub. No.: US 2014/0243652 A1
(43) Pub. Date: Aug. 28, 2014

(54) BODILY SELF-IMAGE AND METHODS FOR PREDICTING PLACEBO RESPONSE OR RESPONSE SHIFT

(71) Applicant: Steven Pashko LLC, Radnor, PA (US)

(72) Inventor: Steven Michael Pashko, Radnor, PA (US)

(21) Appl. No.: 14/351,561

(22) PCT Filed: May 12, 2012
(86) PCT No.: PCT/US2012/038014

§ 371 Date: Apr. 12, 2014

Related U.S. Application Data
(60) Provisional application No. 61/584,735, filed on Jan. 9, 2012.
(60) Provisional application No. 61/535,790, filed on Sep. 16, 2011.

(51) Int. Cl. G06F 19/00; A61B 5/0484; A61B 5/00; A61B 5/0496; A61B 5/04; A61B 5/16; G01R 33/48; A61B 6/03

(52) U.S. Cl. CPC G06F 19/3431; G01R 33/4806; A61B 5/04842; A61B 6/032; A61B 6/037; A61B 5/0496; A61B 5/04001; A61B 5/16; A61B 5/04009; A61B 5/0042
USPC 600/409; 600/300; 600/547; 600/410; 600/425; 600/544; 600/558; 600/407; 705/2

(57) ABSTRACT

Methods for determining the likelihood that a subject will be a placebo responder in a clinical study are provided. Also provided are methods for eliminating likely placebo responders from a clinical study a priori, thereby simplifying data analysis and minimizing or eliminating any confound that arises in the analysis as a result of placebo response. Databases and computer systems using the methods are also disclosed herein. Methods for assessing likelihood of a subject experiencing a response shift are also provided.

### CROSS-REFERENCE TO RELATED APPLICATIONS

This is filed under 35 U.S.C. 371 as a U.S. National Stage of PCT/US2012/038014 filed May 12, 2012, which claims the benefit of U.S. Provisional Patent Application Nos. 61/584,735 filed Jan. 9, 2012, and 61/535,790 filed Sep. 16, 2011, the entireties of each of which are incorporated herein by reference.

### BACKGROUND

1. Field of the Invention

This relates generally to methods for providing improved therapeutic treatments and improved clinical trials for therapeutic treatments. More particularly this relates to methods for predicting placebo response and/or the “response shift” phenomena in people undergoing assessment of health or therapeutic treatment.

2. Description of Related Art

Developing therapeutic treatments and active ingredients for the treatment of specific disease conditions and other maladies is the hallmark of Western-style medicine. Generally prior to the regulatory approval of any proposed treatment, active ingredient, or other therapy, scientific studies to establish the lack of acute toxicity and the long-term safety and efficacy of such treatments are required. Among such studies are human clinical studies or “clinical trials”, which involve separate “phases,” each of which provides data to develop the full picture of the safety and efficacy of the proposed treatment. The complicating effects of the placebo response and response shift can confound analysis of the results of testing any treatment in human clinical trials.

The placebo response or effect has been defined as a therapeutic response to a treatment which is not known to have any actual therapeutic action on the condition for which it is used. A classic example of placebo effect is seen when a patient in a clinical trial receives only a “sugar pill” and yet exhibits a response that is more typical of that expected from patients who receive a therapeutic agent which is known or expected to have activity on the condition.

There are two aspects of the placebo effect that must be considered with respect to therapeutic treatment. The first is the complicating factor for clinical trials. While all of the mechanisms underlying the placebo effect may not be understood, what is known is that some people respond therapeutically to a treatment that does not possess any known therapeutic effect for the condition for which it is given in the trial. Accordingly, the active ingredient or test treatment, which putatively does possess therapeutic action for the condition being treated, needs to be distinguished from the confound of the placebo effect so that the true efficacy and safety of the active ingredient or test treatment can be validly ascertained.

The second of aspect of the placebo effect is that people who respond to placebo or who demonstrate a propensity to ‘response shift’ may be more amenable to lower dosages, improved therapeutic outcomes, higher self-reported perceived improvements, quality of life or the like, as described infra.

There is therefore a need for new methods of conducting or evaluating clinical research and determining appropriate therapeutic treatments.

### SUMMARY

Methods related to measuring or assessing the adaptability or malleability of a person's bodily self-image are provided. The methods have applications for improving clinical trials for therapeutic treatments, for improving data analysis in studies of therapeutic efficacy, and for predicting the propensity for a candidate for a clinical trial to respond to a placebo treatment, and other applications.

Thus, in a first of the several aspects of this disclosure, the inventor has provided methods of selecting participants for a biomedical or health-related research study (“clinical trial”). The methods can also be considered methods of prospectively eliminating from the trial participants who are likely to be placebo responders. The methods generally comprise the steps of:
establishing an inclusion and/or exclusion criterion for the study that encompasses a measure of the participant's propensity to respond to placebo treatment; and
eliminating from the study, a priori, any prospective participant who does not meet the required criteria for inclusion or exclusion.

The inventor has surprisingly discovered that an assessment of the adaptability of participant's perception of their bodily self-image can provide a measure of that person's propensity to respond to placebo treatment. In practice, both the time required for a person to experience a shift in their perception of bodily self-image, as well as the extent of such a perceptual shift may be measured (either in absolute or relative terms). Any direct or indirect measure of this phenomena may be used herein.

The time required for a prospective participant to experience a shift in their perception of their bodily self-image is useful in creating the required inclusion and/or exclusion criterion. Also useful is a determination of the extent of the shift in a subjects' perception of their bodily self-image. Thus, useful measures of the shift in perception may include time to shift, which can be expressed for example as specified time(s) (either as maximum or minimum times) for participants to experience a shift; relative time(s) based on the times for all prospective participants for the study; or time(s), percentage(s) or other measure(s) of a shift in perception of bodily self-image determined from a database comprising assessment data related to adaptability of the perception of bodily self-image from a plurality of people. Other useful measures include assessments of the extent of such a shift, where the extent of the shift may be determined independent of time required for the shift to occur, and may be relative other people, including e.g., other participants in a clinical study, or a population. Measurements or criteria relating to the “extent” of shift may be expressed in various forms for example “to a specified extent,” “greater than a specific extent,” or “less than a specific extent.” As used herein, “to a specified extent” (or “to a specific extent”) indicates any specifications that may be set forth and thus includes “greater than a specific extent” or “less than a specific extent”. For example where an inclusion criterion requires that a subject have the ability to shift “to a specified extent” the requirement in practice may be, e.g., “greater than 50%.”

In another of its several aspects, methods are provided for determining the likelihood that a candidate for a clinical trial will respond to a placebo used in the clinical trial. These methods are meaningful for scientifically clarifying the therapeutic role of a proposed therapy by eliminating or minimizing confounding results, and accordingly are valuable to the pharmaceutical industry and for the regulatory agencies tasked with ensuring that new drugs and other therapeutic treatments are safe and effective. The methods generally comprise the steps of assessing adaptability of the candidate's perception of their bodily self image; and determining the likelihood that the candidate will respond favorably to a placebo based on the assessment.

Because of the potential for added time or expense to qualify a candidate for a clinical study, in some cases it is sometimes useful to first establish that the candidate is otherwise qualified to be a participant in the clinical trial based on the inclusion and exclusion criteria for the clinical trial. It is also useful in some applications of the methods that likelihood of being a placebo responder be used as an additional criterion for inclusion in, or exclusion from, the study.

In another of its several aspects, the invention provides collections of data related to adaptability of perception of their bodily self-image. The collections comprise data for each of a plurality of people. The data are based on, derived from, or obtained during an assessment of the adaptability of the person's perception of their bodily self-image. The assessment of adaptability of the person's perception of their bodily self-image preferably comprises a sensory-perceptual paradox, a computerized assessment tool, a virtual reality effect, an indicia of neurological activity, or an indicia of brain activity. The data may comprise assessments that are time-dependent (e.g. time to shift bodily the perception of bodily self-image) or time-independent (e.g. assessments of the extent of a shift in bodily self-image).

In yet another aspect, the invention provides a novel computerized system, which can be used in structuring in analyzing results of clinical trials. The system comprises:
a collection of data comprising, for each of a plurality of people, data based on, or obtained during, an assessment of the adaptability of the person's perception of their bodily self-image,
one or more data structures for arranging the data,
one or more data storage devices for storing the data,
a database management system adapted for managing the collection of data, and
one or more computers, servers, terminals, or networks for accessing the database management system, data structures, or data.

The data provided herein can be usefully arranged according to the one or more data structures and stored at least temporarily on one or more data storage devices. The skilled artisan will appreciate that the database management system, and one or more of the computers, servers, terminals, or networks are in data communication with each other and/or the data, such that the data can be accessed, managed, queried, or otherwise utilized, for example in connection with clinical trials or planning for therapeutic applications or treatments.

In another aspect of the invention, there are also provided herein are methods of predicting or measuring a person's propensity to respond to administration of a placebo during a clinical trial. The methods generally comprise measuring the time required for the person to experience a shift in perception of bodily self-image, or the extent of such a shift. Such a shift is preferably in response to a sensory-perceptual paradox presented to the person which comprises a visual paradox and stimulation at least one other sense. The other sense is preferably somatosensory or tactile, although any other sense may be used. The methods may comprise verifying or establishing that the time to shift and/or the extent of the shift is/are indicative of propensity to respond to a placebo.

In yet another of the aspects of the invention, provided are methods of improving analysis of data from a clinical trial for a therapeutic treatment. The methods generally comprise the steps of:
(a) obtaining a set of raw clinical data;
(b) evaluating the raw clinical data by standard methods to generate preliminary results;
(c) obtaining the identity for each participant in the trial (i.e. unblinding the study data);
(d) assessing the adaptability each participant's perception of their bodily self-image;
(e) determining which participants have readily adaptable body images;
(f) creating a modified clinical data set by modifying the raw clinical data to identify, eliminate, or statistically adjust data pertaining to those participants determined to have readily adaptable perceptions of their bodily self-images;
(g) evaluating the modified clinical data to generate modified results; and optionally,
(h) using the modified data or modified results in connection with seeking approval for the therapeutic treatment from a regulatory agency.

Assessments of adaptability of a participant's perception of their bodily self-image may comprise a measure of the time to a shift in perception of bodily self-image and/or a measure of the extent of such a shift. The methods provided in this aspect of the invention can optionally further comprise the step of comparing the preliminary results and the modified results to generate a comparison. The comparison can also be used in connection with seeking approval from a regulatory agency.

An additional aspect provides methods of identifying subjects for a therapeutic treatment based on their propensity to respond favorably to a placebo treatment. The methods comprise measuring the ease with which the person can experience a shift in their perception of bodily self-image. Generally, the more easily a person can shift their bodily self-image, the better subject they will be for the therapeutic treatment. The ease of experiencing a shift in perception of bodily self-image can be assessed as a function of time to experience a shift, or as a function of the extent of the shift.

In yet another of its several aspects, the invention provides methods of determining a propensity to experience a response shift in patients with declining health. The methods comprise the steps of assessing adaptability of the patient's perception of bodily self-image; and determining the candidate's propensity to experience a response shift, based on the assessment. The patients are frequently suffering from a terminal, chronic, progressive, or degenerative disease or condition, and/or they may have anxiety, depression, chronic pain, and/or low perceived quality of life.

In a further aspect of the invention, provided are methods of selecting a course of therapy for a patient suffering from a terminal, chronic, progressive, or degenerative disease or condition. The methods comprise the steps of determining which courses of therapy provide an option that might produce a desirable outcome for the patient; for each option, considering the likelihood that the course of therapy will extend the life of the patient, alleviate the suffering of the patient, or otherwise improve the patient's physical or psychological situation; assessing the likelihood that the patient will experience an improved psychological condition due to a response shift; determining the cost-effectiveness for each option; considering any other factors, and selecting a course of therapy for the patient based on the comparison of cost-effectiveness, the likelihood that the patient will experience a response shift and the other factors.

In another aspect of the invention disclosed herein, methods of conducting a quality of life (QOL) study are provided. The methods generally comprise the steps of:
providing a plurality of subjects for the study;
for each subject:
   providing a QOL assessment on each of a plurality, p, of occasions to obtain assessment data over a period of time;
   determining a score or scores for each such QOL assessment;
   determining from the score or scores a baseline QOL response(s), based on the subject's score or scores for an initial number, n, of such occasions; such that p is much greater than n;
   monitoring the subject's score or scores for each subsequent QOL assessment for unexpected deviations from the baseline QOL response; wherein an unexpected deviation is defined as part of the study;
   ascertaining whether there are any known factors that may explain the unexpected deviation from the subject's baseline QOL response;
   if there are no ascertainable factors that explain the unexpected deviation from the subject's baseline QOL response, assessing the propensity of a subject to experience an improved QOL due to a response shift; and
   determining from the testing whether the subject shows a propensity to experience a response shift;
eliminating from the QOL study assessment data from subjects who are determined to show a propensity to experience an improved QOL due to a response shift; and
completing the QOL study or any phase or portion thereof and analyzing the results thereof without the eliminated assessment data.

In a final aspect of the invention, provided are methods for predicting that an individual will be likely to be a placebo responder or will be likely to experience a response shift. The methods comprise obtaining an objective measure of brain activity in the individual, and determining therefrom whether the individual will be likely to be a placebo responder or will be likely to experience a response shift, wherein the measure of brain activity is correlated with performance in an assessment of adaptability of perception of bodily self-image. A database comprising a collection of data useful for establishing a correlation between the objective measure of brain activity and an assessment of adaptability of perception of bodily self-image is also provided.

These and/or further aspects, features, and advantages of the present invention will become apparent to those skilled in the art in view of this disclosure.

### DETAILED DESCRIPTION

Provided herein are methods for improved clinical trials, for determining the propensity of an individual, patient, or candidate for a clinical trial to respond to the placebo effect. Also provided are databases and computer systems useful for determining the likely placebo responders, and for designing improved clinical trials or improving data analysis for data obtained during clinical trials. Methods for identifying patients who will experience psychological improvement from the “response shift” phenomenon are provided as well as methods for determining cost-effectiveness of a course of therapy.

The inventor has surprisingly discovered previously unknown methods for identifying individuals who are likely to respond to placebo or likely to experience the “response shift” phenomenon. While popular notions and traditional Western scientific understandings suggest that self-identity and one's body are inextricably linked, modern researchers have shown that the perception of bodily self-image can apparently be linked to other than one's body. This has been demonstrated experimentally in human subjects by using a rubber arm (see e.g., Botvinick and Cohen, Nature 391, 756 (1998), Armel and Ramachandran, Proc. R. Soc. Lond. B. 270:1499-1506 (2003)), or a mannequin's body (see e.g., Petkova & Ehrsson, PLoS ONE 3(12): e3832 (2008)). More recently, supernumerary limb illusions and body size illusions have also been used to demonstrate the concept (see Guterstam, Petkova, and Ehrsson ‘The Illusion of Owning a Third Arm’ PLoS ONE 6(2): e17208 (2011) and van der Hoort, Guterstam, and Ehrsson ‘Being Barbie: The Size of One's Own Body Determines the Perceived Size of the World’ PLoS ONE 6(5): e20195 (2011)). The rubber arm experiments have been referred to as an “illusion” because those who hold the assumption that the body and the identity are essentially synonymous cannot make sense of it.

In the experiments employing the rubber arm “illusion”, a subject is seated at a desk. Both of the subject's arms are extended straight forward and placed on the desktop, although the left arm is typically pointed more towards one side (e.g. to the left a little). This makes room for a fake arm to be positioned in front of the left shoulder. A barrier is placed between the fake arm and left arm to block the subject's view of their left arm. The experimenter uses a paintbrush to stroke the fake hand while simultaneously stroking the subject's out-of-view left hand.

After 2-3 minutes, most subjects report that the fake hand is their actual hand. They will also point to the fake hand with their right hand when instructed to “Show me your left hand.” Moreover, when the fake hand is threatened physically, as with a hammer, the subjects show signs of nervousness, as measured objectively e.g., by skin conductance recording.

The results of this research demonstrate something that can't be explained using current assumptions about self-identity or bodily self-image. The results show that the link between our body and our identity is either not direct, or the link is at best tenuous and not stable. Within this research paradigm, the visual and tactile senses seem to be major determinants in the decision about just what belongs to one's own body.

The experiments with the mannequin body provide additional information. Human subjects agreed to wear a helmet with a video display inside, allowing them to see only what was displayed on the video screen. The video screen displayed the output (or viewpoint) of cameras in the room. In the first scenario, the cameras were mounted on the head of a standing, unclothed mannequin and pointed downwards to give a full stereoscopic view of the front of the mannequin's body. This experimental setup allowed the subject to only see downward along the mannequin's body. With the subject and the mannequin standing side by side, the experimenter stroked the abdominal area of the mannequin and the subject in unison. The subject could see the mannequin's body being stroked. Further, while the subject could see the stroking of the mannequin's body they could also feel simultaneous stroking downward along their body.

The findings from the first mannequin experiments were that:

1. Subjects reported feeling as if their body had turned into that of the mannequin's, and
2. The subject's reports were confirmed by skin conductance responses, reflecting their nervousness when a knife was used to threaten the mannequin's torso.

More recent related experiments have a used dolls rather than mannequins and yet similar results have been observed; i.e subjects perceive the doll's body as their own.

In a second experimental scenario, the subject again stood with the video display helmet on their head. The experimenter sat in a chair an arm's length in front of the subject with the video cameras attached to a platform on her head. Because the experimenter was in front of him, the subject's video screen showed his own body from the neck down from the front. When the experimenter and subject shook hands, the subjects reported that they felt as if they were in the other person's body and “shaking hands with themselves.” Paraphrasing and using slightly different words, they felt their body was shaking hands with their identity.

Without being bound to any one particular theory of operation, the inventor has noted that this observed flexibility of the perception of bodily-self-image may be explained by considering that the self-identity, or bodily self-image, may actually be a mental concept or a perceptual construct.

The research shows that when our senses are conflicted, e.g. by having the eyes see something from a different point of view than usual or expected, an important clue that links us to identify with our body is lost. By demonstrating that the link between the senses and the body is not fixed or immutable, we can see that our current understanding of the term “self-identity” or “self-image” with respect to the body (sometimes referred to herein as “bodily self-image” is inaccurate. Possible alternative explanations include:

1. The body's senses and perceptions assemble an identity such that it appears to be within our body and/or
2. Self-identity might simply be a term defined by a combination of our own points view.

Kahneman and Riis (2005) postulated two identities in each of us, which they termed the “experiencing self” and the “remembering self”, as a way to explain the observations indicating that “ . . . retrieval and temporal integration of emotional experiences are both prone to error, and that retrospective evaluations are therefore less authoritative than reports of current feeling.”

The “experiencing self” lives fully in each moment but does not keep score about them or sum them into an opinion. It utilizes the immediate “now” as a reference or point of view. The “remembering self” keeps score, maintains records, and summarizes durations of experience into opinions and beliefs.

These constructs fit into a new framework regarding the understanding of identity. This framework suggests that:

1. Either the word “identity” has to be reconceptualized to mean a point of view from which “self” is identified; or
2. Each of us has at least two “selves” within our one body; or
3. Both 1 and 2 are true.

The “remembering self” as presented by Kahneman and colleagues is our operational, work-a-day, identity. In the psychology of the East, it's called the Relative (identity) or the “self” with a lowercase ‘s.’ It's the one most often referred to as “me” by both the average person as well as the psychologically-oriented professionals trained exclusively in the West. It's defined more like a noun and described as in alignment with concepts such as being in a body, having a work role, a societal status, a family network, etc. Other significant characteristics of this type of identity include a sense of linear time, reliance on memories, interest in discursive thought for orientation in the world and use of external anchors as measures of progress and status.

In contrast, the “experiencing self”, also known as the “Self” with an uppercase ‘S’ or ‘no fixed self,’ in the East, is the counterpoint to Kahneman's “remembering self.” Western psychology has yet to fully acknowledge and understand its rightful place. A few noteworthy psychologists have written about what may now be viewed as related aspects of the self. William James, Carl Jung, and Roberto Assagioli, among others, wrote of an identity more fully encompassing than an ego. The experiencing self is purely phenomenological, existing only in the world of direct perception and introspection. It exists only in the present moment prior to evaluations and the layers of conceptualizations that alter its view.

Thus, the inventor has discovered that the ability of a person to shift their perception of their bodily self-image (sometimes referred to herein as the “adaptability,” “flexibility,” or “malleability” of one's perception of bodily self-image) correlates well with being a placebo responder or response shifter. More specifically, the more easily, the more quickly, or the more extensively or more completely one can shift their personal perception of their own bodily self-image, the more likely they are to be placebo responders in a clinical trial, or to experience a response shift. This discovery enables one to improve clinical trials and therapeutic treatment, for example, by avoiding, a priori, the inclusion of placebo responders in clinical trials to facilitate cleaner efficacy studies, or by processing clinical data, ex post facto to remove confounding placebo responders who are identified in a simple, separate, and objective manner.

### Definitions & Abbreviations

Unless expressly defined otherwise, all technical and scientific terms, terms of art, and acronyms used herein have the meanings commonly understood by one of ordinary skill in the art in the field(s) of the invention, or in the field(s) where the term is used. In accordance with this description, the following abbreviations and definitions apply.

As used herein, the singular form of a word includes the plural, and vice versa, unless the context clearly dictates otherwise. Thus, the references “a”, “an”, and “the” are generally inclusive of the plurals of the respective terms. For example, reference to “a trial” or “a participant” includes a plurality of such “trials” or “participants.”

The words “comprise”, “comprises”, and “comprising” are to be interpreted inclusively rather than exclusively. Likewise the terms “include”, “including” and “or” should all be construed to be inclusive, unless such a construction is clearly prohibited from the context. Further, forms of the terms “comprising” or “including” are intended to include embodiments encompassed by the phrases “consisting essentially of” and “consisting of”. Similarly, the phrase “consisting essentially of” is intended to include embodiments encompassed by the phrase “consisting of”.

Where used herein, ranges are provided in shorthand, so as to avoid having to list and describe each and every value within the range. Any appropriate value within the range can be selected, where appropriate, as the upper value, lower value, or the terminus of the range.

The methods and devices and/or other advances disclosed here are not limited to particular methodology, protocols, and/or structures described herein because, as the skilled artisan will appreciate, they may vary. Further, the terminology used herein is for the purpose of describing particular embodiments only, and is not intended to, and does not, limit the scope of that which is disclosed or claimed.

Although any devices, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent permitted under applicable law. Any discussion of those references is intended merely to summarize the assertions made therein. No admission is made that any such patents, patent applications, publications or references are prior art, or that any portion thereof is either relevant or material to the patentability of what is claimed herein. Applicant specifically reserves the right to challenge the accuracy and pertinence of any assertion that such patents, patent applications, publications, and other references are prior art, or are relevant, and/or material.

As used herein a “placebo” refers to any therapy used by a person to obtain a purported, supposed, or believed therapeutic effect on a symptom, disorder, condition, or disease, or prescribed, recommended, endorsed or promoted, knowingly or unknowingly, to another, notwithstanding that the therapy is actually ineffective for, has no known physiologic effect on, or is not specifically effective for the symptom, disorder, condition, or disease being treated.

The “placebo effect” and “placebo response” as used herein are interchangeable and refer to any non-specific, psychological, psychotherapeutic, or unexplained physiological effect produced by a placebo, or the effect of spontaneous improvement attributed to placebo. Placebo response is used frequently defined operationally in clinical trials and accordingly the precise determination of a “placebo response” varies according to the aims of a specific research study. At times, it is more broadly defined, so that more people will fall into the group. This requires the active drug treatment to be more efficacious in order to show a statistically significant clinical effect of the therapeutic treatment being tested.

The term “response shift” as used herein refers to a change in the meaning of one's evaluation of a construct as a result of a change in one's internal standards of measurement, a change in one's values, or a change in one's definition of the construct. Response shifts may be observed in patients with terminal, chronic, progressive, degenerative, and/or deteriorating conditions or diseases. Examples, include chronic pain (including neuropathic pain), neurodegenerative diseases, and cancer, as well as anxiety, depression, or the like. Response shift can be a confounding factor in quality of life studies and related studies where subjects self-report their evaluation of various constructs, assessments, questions, or the like. In some views a response shift can be seen as similar or identical to a placebo effect (or even a generalized form thereof) (Wilson, 1999), where the main difference is that no placebo is administered and yet the subject experiences at least a perceived improvement in condition. The above definition is consistent with that provided in Sprangers M A, Schwartz CE: “Integrating response shift into health-related quality of life research: a theoretical model.” Soc. Sci. Med 1999, 48:1507-1515, which is specifically incorporated herein by reference.

As used herein “bodily self-image,” and “self-identity” are generally synonymous and refer to an individual's perception of their own self in relation to, or in relationship with their body. Thus, if under experimental conditions an individual has a physiological or psychological response to a perceived threat to, e.g., a rubber arm or a mannequin's torso, that individual can be understood to have perceived the rubber arm or mannequin as part of their body, meaning their bodily self-image has shifted from what they otherwise “know” is their physiological body to the “other” body or body part. Such a shift can occur even where the person otherwise would clearly intellectually recognize that the “other” body or body part cannot be “self” e.g. where the body or body part is positioned at an impossible location or comprises an impossible object (such as a block of wood rather than an arm, or even space itself) or any similar construct such as a supernumerary limb, or an impossible body (in terms of size, shape, composition, or the like).

The terms “adaptability,” “flexibility,” and “malleability” are used synonymously and indicate an ability to change or shift, e.g. from one bodily self-image to another. Adaptability is neither inherently desirable or undesirable, however the measure of such adaptability it may be used in different ways. In some embodiments herein, adaptability is used to exclude a subject from a clinical trial. In other embodiments, it is used to identify a subject or candidate for certain therapeutic treatments. In yet other embodiments, adaptability is used to identify people with a propensity to be placebo responders or likely to experience a response shift. Assessments of adaptability may include time-based assessments (i.e. assessments directly or indirectly incorporating time as a measure of the response or an indicator of ability to shift bodily perception). Assessments of adaptability may also be completely independent of time, for example assessments that only consider the extent of a shift under a particular set of conditions or in a given scenario.

In certain embodiments herein, the ability to shift the perception of bodily self-image (i.e., the ability for one to experience a shift in their perceived bodily self-image) can be determined or measured using an “objective measure.” Any objective measurement capable of measuring a physiological (including neurological) or psychological parameter may be used herein, including any measure of a perceptual or cognitive process. Such objective measures may or may not be physically or electrically connected to a subject's physical body (including without limitation the torso, head, limbs, and/or extremities). In some embodiments, an objective measure may comprise a video or digital recording of the subject, including for example, their facial expressions, and/or eyes. Examples of such objective measures include heart rate monitoring, blood pressure monitoring, monitoring respiration, measuring one or more components of blood (e.g. blood chemistry) or other bodily fluid, measuring skin parameters such as blood flow, temperature, or conductance; or other physiological measures including measuring any brain or neurological activity. Like other assessments, objective measures may be dependent or independent of time for a subject to experience a shift. Objective measures also include the use of instruments, such as those for any one or more of skin conductance resonance (SCR), electroencephalography (EEG), quantitative EEG (QEEG), magnetic resonance imaging (MRI), functional MRI (fMRI), computed tomography (CT), positron emission tomography (PET), electronystagmography (ENG), single photon emission computed tomography (SPECT), magnetoencephalography (MEG), superconducting quantum interference devices (SQUIDS), electromyography, eye movement tracking, and/or pupillary diameter change.

As used herein a “clinical trial” or “clinical study” is any research study, such as a biomedical or health-related research study, designed to obtain data regarding the safety or efficacy of a therapeutic treatment such as a drug, device, or alternative treatment. Such studies can be conducted to study fully new drugs or devices, new uses of known drugs or devices, or even to study old or ancient treatments that have not been used in Western-style medicine or proven effective in such studies. Clinical studies frequently include use of placebo treatments for one group of subjects. Clinical studies are in some embodiments conducted as double blind studies wherein the subjects do not know whether they received a putative active ingredient or treatment for the condition being tested, or a placebo with no known physiologic effect on the condition. In addition, in such double-blind studies, the researchers collecting the data also do not know which subjects received placebo or active treatment. Double blind studies help prevent bias for or against the test treatment. Moreover, while the use of placebos can help prove the efficacy of new drugs, if a research study turns out to include many people who respond to the placebo, it is much more difficult to establish the efficacy of what may well be a worthwhile therapeutic compound.

A “candidate” or “prospective participant” for a clinical trial means a person who is being considered for enrollment in the study, subject to satisfying the trial's approved inclusion and exclusion criteria. Thus the pool of candidates or prospective participants is necessarily larger than the number of “participants” required for participation in the study. As described in detail below, various embodiments of the methods provided herein may be either applied prospectively, e.g. by assessing candidates or prospective participants, or applied after-the-fact by assessing participants enrolled in a particular study, whether before, during, or after the completion of the clinical study. A person being assessed using the methods herein may be referred to as a candidate, a prospective participant, a participant, a subject or similar terms. Distinctions will be clear to the reader from the context of a given description.

As used herein “quality of life” refers to any measure of influences upon the goodness and meaning in life, a person's happiness and/or well-being, or the degree to which a person enjoys the important possibilities of his or her life and can consider such factors as whether one subjectively believes that they have, can, or will achieve their personal goals, hopes, and aspiration, or how one subjectively feels connected to or in control of one's environment. Quite literally, quality of life (QOL) studies can relate to man's search for meaning. Quality of life can be measured in terms of how one's life is negatively affected (on an individual level) by, for example, health concerns including fears about health, any chronic, degenerative, progressive, or end-stage disease processes, a debilitating illness that is not life-threatening, life-threatening illness that is not terminal, terminal illness of any kind, the predictable, natural decline in the health of an elder, the mental and/or physical decline of a loved one, and even conditions that have symptoms but which have defied diagnosis. In addition to health or wellness factors, QOL studies can also be impacted by economic, political, and even environmental factors. Various researchers have developed “Quality of Life” models based on criteria they deem important. Examples include the EuroQOL and others. The skilled artisan can select any such models that may be found useful for purposes herein.

### Abbreviations

The following abbreviations apply unless indicated otherwise:
ALS: amyotrophic lateral sclerosis;
CCTV: closed circuit television;
CD: compact disc;
CD-ROM: compact disc read-only memory;
cm: centimeter(s);
CT: computed tomography;
EEG: electroencephalogram;
ENG: electronystagmography;
fMRI: functional MRI;
HAM-A: Hamilton Anxiety Scale;
HAM-D: Hamilton Depression Scale;
HMD: head-mounted display;
HUI-3: Health Utility Index, Mark III;
IMMPAC: Initiative on Methods, Measurement, and Pain Assessment in Clinical Trials;
MEG: magnetoencephalography;
MD: muscular dystrophy;
mho: seimens;
MRI: magnetic resonance imaging;
MS: multiple sclerosis;
NDA: new drug application;
NIH: National Institutes of Health;
PET: positron emission tomography;
QEEG: quantitative EEG;
QOL: quality of life;
s: seconds;
SCR: skin conductance response;
SPECT: single photon emission computed tomography;
SQUIDS: superconducting quantum interference devices;
t: time;
u: micron(s).

### DETAILED DESCRIPTION OF ILLUSTRATIVE EMBODIMENTS

In a first of it several aspects, provided herein are methods of selecting participants for a biomedical or health-related research study (“clinical trial”) comprising the steps of:
(a) establishing at least one inclusion and/or exclusion criterion for the study that encompasses a measure of the participant's propensity to respond to placebo treatment;
(b) eliminating, a priori, from the study any prospective participant who does not meet the required criteria for inclusion or exclusion;
wherein the measure of propensity to respond to placebo treatment comprises an assessment of the adaptability of participant's perception of their bodily self-image.

In one embodiment of the method, at least one exclusion criterion excludes prospective participants who, in a specified assessment, have the ability to shift their perception of their bodily self-image within a specified time(s). Thus, the researcher may establish a criterion that is essentially a cut-off time whereby if the prospective participant experiences a shift in their perception of bodily self-image in less than the specified time, they are excluded from the clinical study. Alternatively, an exclusion criterion may specify relative time(s) based on the times for all prospective participants for the study. For example, the exclusion criteria may exclude anyone who tests within the fastest 20% of all prospective participants in the study. While about 20% may be a useful relative time based on observations to date, the percentage may understandably vary. In various embodiments, the exclusion criteria may exclude the fastest 35%, 30%, 25%, 22.5%, 20%, or less of all prospective participants. In other embodiments, the fastest 17.5%, 15%, 12.5% or even 10% are excluded from the study. The skilled artisan will appreciate that such exclusion criteria may vary from study to study depending on the pool of prospective participants, the population being tested in the study, and other factors. Moreover the skilled artisan will understand that while whole numbers are easier to work with and more convenient, data may dictate that other than whole percentages be used. Thus, all numbers and ranges between the stated numbers are intended—e.g. a range of 20% to 22% would include 20.5% 21.8%, and so on.

In another embodiment of the method, at least one exclusion criterion excludes prospective participants who, in a specified assessment, have the ability to shift their perception of their bodily self-image to a specified extent. The extent of such a shift may be defined by the researcher in a variety of ways, discussed below. As with the time-based criteria, the researcher may establish a criterion that is essentially a cut-off whereby if the prospective participant experiences a shift in their perception of bodily self-image to an extent greater than that specified by the researcher, they are excluded from the clinical study.

To assess the extent of a shift in perception of bodily self-image, a subject's responses to questions, for example about their health status or health preferences, can be determined with the subject in an initial state and a subsequent state (e.g., before and after being presented with an assessment such as a sensory-perceptual paradox). A comparison can be made, and based on changes in the subject's answers, the subject can be assigned a health status score which can be used to compare different subjects. One important purpose of the comparative questioning or testing is to determine if the subject's health ‘status’ changes because of a shift in the subject's perception of bodily self-image.

The questions can be provided to a participant or subject informally or more formally, and may include questions about a variety of topics. Presently preferred are questions related to the subject's health, psychology, emotion, interest level, outlook, motivation, pain, sensory experience, or the like. Questions may be presented orally, or through a paper and pencil-type instrument, a standardized test, electronically, or in a audio/video recording. Standardized tests suitable for use herein include those designed to determine whether someone is suffering from anxiety, depression, QOL, or the like, and may include verbal or written reports from unstructured questions, and/or revealed preferences (e.g. the subject chooses to do some action and in doing so they are observed to make choices that reveal their preferences). The subject's responses to such questions can be recorded to facilitate before and after comparisons, as well as scoring.

Generally the subject's initial responses to the set of questions will be obtained prior to the presentation of the assessment. The initial and subsequent responses can be obtained on the same or on different days. An advantage of obtaining responses on different days is to avoid any bias, carry-over effects, confounding of responses, or the like. Thus, in some embodiments, the assessment (e.g. a sensory-perceptual paradox) may be presented first, then the questions can be asked to solicit responses thereafter, since the ‘before’ responses can be obtained on a different day.

Preferably the assessment, such as a sensory-perceptual paradox is standardized for all subjects in a given study so that subjects can more readily be compared with respect to the extent of a shift. In one embodiment, the exact same wording and presentation of the sensory paradox are retained from subject to subject. In some embodiments, objective measures such as skin conductance can be used to confirm a shift has occurred, or can be used to correlate the extent of the shift in some embodiments. Once shifted, the subject's responses to the questions (also preferably standardized) are obtained for comparison with the subject's responses in the absence of the paradox. In these embodiments, the assessment is geared primarily to ascertaining the extent of a subject's shift in perception of bodily self-image. Such measures are generally time-independent, however, to facilitate ease of testing, the assessment may be standardized for a set period of time, and/or may be terminated after a given degree of shift is achieved, e.g. as determined by an objective measure such as skin conductance.

In various embodiments, an exclusion criterion will entail time(s), percentage(s), extent(s) and/or other measure(s) of a shift in perception of bodily self-image, including those determined from a database comprising assessment data related to adaptability of the perception of bodily self-image from a plurality of people. In such embodiments, the database may permit comparisons among similarly situated participants from other studies collected over a period of time, or may permit comparisons based on age, gender, education level, occupation, health status, condition being treated, and/or other factors that will allow more accurate comparisons and determinations of the ability to shift perception of bodily self-image. In one embodiment, the researcher specifies an exclusion criterion that relates to both of time and extent of a shift in perception of bodily image, both of which may be determined in a single assessment, or in multiple assessments.

In yet other embodiments, rather than or in addition to an exclusion criterion, an inclusion criterion can be formulated for the study. Such an inclusion criterion can require that, in a specified assessment, participants do not shift their perception of their bodily self-image faster than a specified time. For example a criterion could provide that to be included in the study a prospective participant must not experience a shift in perception of bodily self-image perspective in less than 1 minute, or less than 30 seconds, or 10 seconds.

In other embodiments, an inclusion criterion may specify that participants must not shift their bodily self-image faster than a relative time based on the times for all prospective participants for the study, For example participants must be in the faster 25% of all prospective participants. As with the exclusion criteria, depending on the study and the requirements, the specific percentages can vary from about 35%, 30%, 25%, 22.5%, 20%, 17.5%, 15%, 12.5%, 10% to less than about 10%, with all numbers and ranges therebetween included.

As with the exclusion criteria, an inclusion criterion can state the requirements as time faster than a defined time, or within a percentage, or other measure determined from a database comprising assessment data related to adaptability of the perception of bodily self-image from a plurality of people.

Inclusion criteria can also be related to the extent of a subject's permissible shift in perception of bodily self-image. For example, an inclusion criterion could require that a subject not shift more than some stated amount expressed a score, percentage, or other meaning number comparing one particular subject to a population or group of subjects. E.g. a subject in the 76-99 percentile in terms of extent of shift would automatically not satisfy an inclusion criterion that required a subject to be in no higher than the 75 percentile.

The assessment of adaptability of the perception of bodily self-image in a participant can comprise a sensory-perceptual paradox, a computerized assessment tool, a virtual reality effect, an indicia of neurological activity, or an indicia of brain activity.

The sensory perceptual paradox (sometimes referred to herein as a sensory paradox) requires that at least one and preferably at least two senses are implicated in the assessment. While for present purposes this is referred to as a paradox, as discussed above some assessment have been termed “illusions.” The distinction may be semantic, and the skilled artisan will recognize that the assessment is paradoxical in that the subject may fully understand at one level that the rubber arm or such is not “self,” and yet at another level the subject experiences one or more aspects of “self” in the rubber arm or whatever is presented during the sensory paradox.

In one presently preferred embodiment, the assessment comprises use of a sensory-perceptual paradox that involves stimulation of the visual sense, a visual illusion, visual effects, or digital imagery. The visual sense is a powerful sense in establishing and/or determining one's perceptions and perspectives. While the visual aspects of the sensory perceptual paradox can be created entirely manually, for convenience, reproducibility, control, and variety, in various embodiments the sensory paradox is at least partially created using electronic equipment, a computer processor, or a digital medium. The sensory-perceptual paradox preferably involves at least one other sense, wherein the visual sense and other sense are simultaneously or nearly simultaneously stimulated as part of the paradox.

The other sense is somatosensory or tactile in certain embodiments, although other sense may be used.

As discussed above with respect to the rubber arm or the mannequin experiments, the sensory-perceptual paradox in some embodiments comprises a simulated or artificial body or body part. These can follow the structure of the rubber arm or mannequin experiments, and/or the variations described herein. In one embodiment, stimulation of the visual sense comprises allowing the person to view the simulated or artificial body or body part, and stimulation of the somatosensory sense comprises simultaneously touching the simulated or artificial body or body part and the corresponding actual body or body part. In other embodiments, the subject is instructed to move (e.g. wiggle, bend, flex, etc.) the corresponding body or body part, and the simulated or artificial body or body part is moved in sympathy with the instructions, either by an experimenter, through a computer or the like (e.g. robotically or animatronically), or through a virtual reality environment or the like.

In various embodiments of these sensory-perceptual paradox scenarios the simulated or artificial body or body part
a) is lifelike and presents a less extreme paradox to the participant's perception; or
b) is not lifelike and presents a more extreme paradox to the participant's perception.

In yet other embodiments, the paradox is made more intense or more extreme by altering the positioning in space of the simulated or artificial body or body part. For example, a body part can be placed at more and more paradoxical distances or angles from the subject such that it should be harder for the subject to shift the perception of their bodily self-image relative to the simulated or artificial body or body part. The time required to make a shift in perception will generally increase as the paradox construct presented is increasingly extreme, intense, or paradoxical.

In another of its several aspects, methods are provided herein for determining the likelihood that a candidate for a clinical trial will respond to a placebo used in the clinical trial. The methods generally comprise the steps of assessing adaptability of the candidate's perception of bodily self image; and determining the likelihood that the candidate will respond favorably to a placebo based on the candidate's response to the assessment.

In some embodiments it is advantageous to pre-qualify the candidates based on the inclusion and exclusion criteria for the study before assessing the qualifying candidates to determine whether they are a likely placebo responder. In other embodiments, the likelihood of being a placebo responder can be used as an additional criterion for inclusion in or exclusion from the study, as discussed above with respect to the first aspect of the invention.

The assessment of the adaptability of the candidate's perception of bodily-self image preferably comprises a sensory-perceptual paradox, a computerized assessment tool, a virtual reality effect, an indicia of neurological activity, or an indicia of brain activity. Presently preferred for use with the methods are sensory-perceptual paradox comprising stimulation of the visual sense. Such paradoxes may include a visual illusion, visual effects, or digital imagery.

Preferably, the sensory paradox involves at least one other sense, wherein the visual sense and other sense are each stimulated as part of the paradox. In several embodiments, the visual and other sense are stimulated simultaneously or nearly simultaneously. In some embodiments, a delay is introduced, i.e., the time between stimulation of the two senses is purposely by increased thereby increasing the degree of the paradox, i.e making it more paradoxical. The skilled artisan can readily determine the maximum delay where the subject is not able to shift because the perception of the delay in stimulation overwhelms or overtakes any tendency to shift the perception of bodily-self-image.

Because of the potential advantages and ease of use of technology in some embodiments the sensory paradox is at least partially created or displayed to a subject using a digital medium or a computer processor.

In one embodiment, the other sense is the somatosensory or tactile sense. In yet another embodiment, it is a kinesthetic or “felt” sense.

The likelihood that the candidate will respond to a placebo can preferably be expressed or determined as a function of the time, duration, intensity and/or extent, or any combination thereof, of the candidate's response to the assessment, e.g. the time required to shift perception of bodily self-image in response to a sensory-perceptual paradox may be a useful proxy for the likelihood to be a placebo responder. Independent of the time, the extent to which a candidate can make a shift in perception of bodily-self image may also serve as a useful proxy for placebo response—i.e. candidates that can more extensively or more fully make the shift have a greater likelihood of being placebo responders or response shifters.

The candidate's response to the assessment can be determined from an objective measure of neurological activity or brain activity. The measure can be a dynamic or static image or series of images of the candidate or candidate's brain activity, or other measure of neurologic activity. Examples of objective measures of such activity that may be suitable for use herein include electroencephalography (EEG), particularly QEEG, magnetic resonance imaging (MRI), particularly fMRI, computed tomography (CT), positron emission tomography (PET), electronystagmography (ENG), single photon emission computed tomography (SPECT). Evoked potentials, may also be useful as an objective measure.

Another type of objective measure useful herein is related to skin conductance, for example the methods known variously as skin conductance response (SCR), galvanic skin response (GSR), electrodermal response (EDR), psychogalvanic reflex (PGR), or skin conductance level (SCL).

While the inventor presently prefers providing a sensory-perceptual paradox as the ‘gold-standard’ for assessing the flexibility or adaptability of a subject's perception of bodily self-image, the skilled artisan will appreciate that correlations may be established between performance with respect to the sensory-perceptual paradox and one or more quantitative or objective measures of neurological activity or brain activity. Thus, it is expected that as these correlations develop more fully, that it may actually be preferred to simply use an objective measure in the first instance to predict whether a subject is likely to be a placebo responder.

Another aspect of the invention provides a collection of data comprising, for each of a plurality of people, data based on or obtained during an assessment of the adaptability of the person's perception of their bodily self-image.

The assessment of adaptability of the person's perception of their bodily self-image preferably comprises a sensory-perceptual paradox, a computerized assessment tool, a virtual reality effect, an indicia of neurological activity, or an indicia of brain activity. The data comprise one or more of the time of the candidate's response to the assessment, the duration of the candidate's response to the assessment, the intensity and/or extent of the candidate's response to the assessment, or any combination thereof. As with all of the aspects disclosed herein, the time, duration, intensity and/or extent can be determined from for example a paper and pencil instrument that is designed to ascertain the candidate's experience, or from oral self-reports from the candidate during the presentation of the sensory-perceptual paradox, or obtained thereafter. The time, duration, intensity and/or extent can also be determined from an objective measure as discussed above. It will be noted that discussions herein and throughout the disclosure about ways to measure or evaluate the assessments of adaptability, and/or the likelihood or the propensity of a subject being a placebo responder or a response shifter, may be applied to any of the aspects of the invention unless expressly excluded.

Thus, in one embodiment, the data comprise an objective measure of neurological activity, brain activity, or skin conductance related to an assessment of a person's perception of their bodily self-image, as described above.

In another aspect, provided are computerized systems comprising:
a collection of data comprising, for each of a plurality of people, data based on or obtained during an assessment of the adaptability of the person's perception of their bodily self-image,
one or more data structures for arranging the data,
one or more data storage devices for storing the data,
a database management system adapted for managing the collection of data, and
one or more computers, servers, terminals, or networks for accessing the database management system, data structures, or data.

The skilled artisan will understand that the data can be arranged according to the one or more data structures and stored at least temporarily on one or more data storage devices. The database management system, and one or more of the computers, servers, terminals, or networks are in data communication with each other and/or the data. The system thus allows new data to be written/saved, data to be modified, stored, accessed, queried, revised, or otherwise operated on, as needed. The system can provide different levels of access or different privileges to each of a plurality of users, such that the database remains secure, and yet the users can design queries to explore the data and develop new understandings of the underlying phenomena by further research into the data. The computer system and the collection of data described herein and above become more powerful as the data set grows, and more data are obtained. The data may include all of the information, results, and statistics from a plurality of clinical trials wherein the assessments disclosed herein are applied. Thus the database and the computer system facilitate, for example, the establishment of stronger and better correlations between a subject's actual degree of response to a placebo in a clinical trial, and the assessment of the adaptability of that subject's perceptions regarding bodily self-image. The computer system and collection of data are equally useful for establishing correlations between the assessments and likelihood of experiencing a response shift.

The collection of data can further include other data, whether or not pertinent to the clinical trial originally conducted, such as age, gender, educational background, occupation, interests etc. Any such data should of course be maintained in a way to protect the privacy and identity of those included, and only be obtained and used with proper permission.

The computerized system can further comprise a set of inclusion or exclusion criteria for a clinical trial. Preferably at least one criterion in the set is based on or derived from the collection of data, and is either directly or indirectly dependent on the specific data related to an assessment of adaptability of a subject's perception of their bodily self-image.

In another of its several aspects, provided are methods of measuring a person's propensity to respond to administration of a placebo during a clinical trial. The methods comprise the steps of measuring the time required for the person to experience a shift in perception of bodily self-image, and/or the extent of that shift in response to a sensory-perceptual paradox comprising a visual paradox and stimulation at least one other sense, and determining if the time to shift and/or extent of the shift is indicative of propensity to respond to the placebo. In presently preferred embodiments, the other sense is somatosensory, tactile, or kinesthetic.

In one embodiment the sensory-perceptual paradox comprises simultaneously or nearly simultaneously stimulating the person's visual and somatosensory senses. The propensity to respond to placebo administration can be conveniently determined via self-reported responses from the person, or answers on a paper and pencil instrument. The propensity to be a placebo responder is generally inversely related to the time required to shift body image perception, and generally directly correlated with the extent of the shift.

In one embodiment, the sensory-perceptual paradox comprises a simulated or artificial body or body part corresponding to an actual part of the person's body. Stimulation of the visual sense comprises allowing the person to view the simulated or artificial body or body part in such embodiments. The methods further comprise stimulation of the somatosensory sense, for example simultaneously or nearly simultaneously touching the simulated or artificial body and the corresponding actual body part. The actual part of the person's body kept out of the person's visual field while the simulated or artificial body or body part is visible.

A shift in perception is determined by a psychological measure or a physiological measure, or is determined subjectively by oral self-reporting by the person, or by answers to a paper and pencil assessment of the paradox. In some embodiments determinations are made before and after presentation of the paradox. In one embodiment a shift in perception is determined by an objective measure of neurological activity, brain activity, or skin conductance, for example the objective measure is obtained via skin conductance resonance (SCR) or an equivalent thereof, electroencephalography (EEG), magnetic resonance imaging (MRI), computed tomography (CT), positron emission tomography (PET), electronystagmography (ENG), single photon emission computed tomography (SPECT) or by measuring evoked potentials.

Another aspect of the invention provides methods of improving data analysis for data from a clinical trial for a therapeutic treatment. The methods are generally for the ex post facto analysis of data from clinical trials where likely placebo responders were not eliminated from the clinical trial a priori. One benefit of the present methods is that they provide solutions for resolving confounding placebo effects in clinical data whether they are applied beforehand or after the fact of the actual trial. For many reasons, the a priori methods may be more efficient and more economical than the after the fact methods, however, for data sets that already have been collected, or for clinical trials in progress, analyzing the data set that includes placebo responders may be more useful.

Thus, the methods in connection with this aspect of the invention comprise the steps of:
(a) obtaining a set of raw clinical data (which generally include one or more placebo responders);
(b) evaluating the raw clinical data by standard methods to generate preliminary results;
(c) obtaining the identity for each participant in the trial (i.e. unblinding the study data);
(d) assessing the adaptability each participant's perception of their bodily self-image (e.g. obtaining a measure of the likelihood that that participant is a placebo responder);
(e) determining which participants have readily adaptable body images;
(f) creating a modified clinical data set by modifying the raw clinical data to identify, eliminate, or statistically adjust data pertaining to those participants determined to have readily adaptable perceptions of their bodily self-images;
(g) evaluating the modified clinical data to generate modified results; and optionally,
(h) using the modified data or modified results in connection with seeking approval for the therapeutic treatment from a regulatory agency.

The skilled artisan will appreciate that step (a) is a prerequisite to the method, in that the method cannot be applied until clinical trial data are available, e.g. a clinical trial is either complete, or underway to at least the point of an initial data collection. It is to be understood that step (b), i.e. evaluating the data by standard methods is not essential to the method and may be eliminated however, it is believed it will be generally employed by the researchers or analysts and generally expected by regulators.

In step (f), data pertaining to those participants determined to have readily adaptable perceptions of their bodily self-images are identified, eliminated, or statistically adjusted to account for the fact that these were likely placebo responders during the clinical trial. The skilled artisan will understand that the data modified (identified, eliminated, or statistically adjusted) will be those related to the clinical trial for those participants. Data that would not be modified would include data not related to likely placebo responders. Also not modified would be the collected data and basic factual information relating to likely placebo responders (e.g. raw data would remain intact).

Data that may be modified would include response data to the therapeutic treatment or placebo. The least preferable modification is to merely identify suspect data that comes from likely placebo responders, for example with a series of footnotes or other explanatory notes. If the data for likely placebo responders can be eliminated from the data set without compromising the integrity of subsequent statistical analyses, that may be most preferred. Alternatively, data for likely placebo responders may be statistically adjusted, for example by weighting the data for subjects who are less likely to respond to placebo more heavily than the data for likely placebo responders. Statistical models are available and skilled artisans will be readily able to apply appropriate or suitable statistical adjustments to the collected data to allow the modified data set to be created.

A much clearer picture of therapeutic efficacy of a treatment may emerge from the study or analysis of the modified clinical data as compared to the understanding that comes from the raw data. By eliminating or adjusting for the likely placebo responders, confounding effects may be removed.

In some embodiments, the methods comprise a further step of comparing the preliminary results and the modified results to generate a comparison, and optionally using the comparison in connection with seeking approval from a regulatory agency.

As with other aspects disclosed herein above, the steps of assessing the adaptability each participant's bodily self-image and determining which participants have readily adaptable bodily self-images comprise one or more of a sensory-perceptual paradox involving at least two senses, a computerized assessment tool, a virtual reality effect, a simulated or artificial body or body part; or a psychological or physiological measure of a shift bodily self-image perception.

Yet another aspect of the invention provides methods of identifying subjects for a therapeutic treatment based on each subject's individual propensity to respond favorably to a placebo treatment. The methods comprise the step of measuring the ease with which the person can experience a shift in their perception of bodily self-image.

In general for this aspect of the invention, the more easily a person can shift their bodily self-image, the better a subject they will be for the therapeutic treatment. The ease with which a person can experience a shift in the perception of bodily self-image can be expressed as a function of the time required for the person to experience a given shift, the duration of the shift experienced, the intensity and/or the extent to which the person experiences a shift.

In one embodiment, the person/subject is presented with a sensory-perceptual paradox, and the ease with which the person/subject can experience a shift in the perception of bodily self-image is a function of the degree of paradox presented to the person.

In presently preferred embodiments of this aspect, the sensory-perceptual paradox comprises stimulation of the visual sense by allowing the person to view a simulated or artificial body or body part, and further comprises stimulation of the somatosensory by simultaneously or nearly simultaneously touching the simulated or artificial body and the corresponding actual body or body part. To make the paradox more acceptable, the person's corresponding actual body or body part is precluded from the person's visual field while the simulated or artificial body or body part is visible.

The degree, magnitude, or severity of the paradox presented can be a function of how life-like the simulated or artificial body or body part is; i.e. the more life-like the simulated body or body part is the lower the degree of paradox and the less life-like the simulated body or body part is, the higher the degree of paradox. The degree of paradox can also generally relate to the position in which the simulated or artificial body or body part is presented, for example the angle of presentation, or the distance of presentation. When a simulated or artificial body or body part is presented a less probable, improbable or even impossible angle or distance, the degree, magnitude or severity of paradox is increased. However, it is possible for a subject to experience a shift in the perception of their bodily self-image even when the simulated or artificial body or body part is presented at an impossible angle or an impossible distance relative to the subject.

For this aspect of the invention, the therapeutic treatment comprises for example a modified or reduced dosing regimen, a modified or reduced time of therapeutic treatment, a therapeutic treatment with fewer side effects than a standard of care therapy, an alternative to a standard of care therapy, or a placebo.

Because the method is selecting for likely placebo responders and/or response shifters, it is expected that for certain therapeutic treatments with active ingredients, lower dosages, shorter time courses, and/or lower circulating blood levels of active ingredient, or the like may work as well or provide the same clinical benefits in the likely placebo responders and response shifters as higher doses, longer time courses, and/or higher circulating blood levels of active ingredient work in non placebo responders/nonresponse shifters. Because populations of likely placebo responders and/or response shifters could not previously be determined a priori, it was not possible to consider the benefits that could accrue to this population such as reduced side effects, reduced exposure time, reduced clearance periods, as well as the potential benefits for medical providers of reduced costs for such populations. Surprisingly, as a result of the inventor's discovery, clinical trials designed to test such hypotheses are now possible.

Such methods may have particular benefits where a subject is suffering from a health-related condition comprising anxiety, or depression or an anxiety-related or depression-related disorder, a neuropathy, or chronic pain and where the therapeutic treatment is for treating the condition. Since likely placebo responders and/or response shifters are more likely to notice and/or report improvements in their personal state of anxiety, depression, or pain (in theory by being more readily in the “experiencing self”)—it is expected that these and related types of conditions would be well suited to therapeutic treatment according to the method.

In yet another aspect of the invention, methods are provided for determining, in patients with declining health, a propensity to experience a response shift, the method comprising the steps of assessing adaptability of the patient's perception of bodily self-image; and determining the candidate's propensity to experience a response shift, based on that assessment.

In one embodiment, the patient is suffering from a terminal, chronic, progressive, or degenerative disease or condition. In other embodiments, the patient suffers from one or more of anxiety, depression, chronic pain, progressive degeneration of any physical or mental function, or low perceived quality of life (QOL).

The methods are particularly useful where the disease or condition causes an impairment or loss of function of the central nervous system, peripheral nervous system, brain, heart, lungs, circulatory system, bones, joints, pancreas, kidneys, immune system, or any combination thereof. Examples of such include any terminal cancer or other condition, a neurodegenerative condition, a spinocerebellar ataxia, an encephalopathy, or other condition causing cerebellar degeneration, congestive heart failure, a muscular dystrophy, cirrhosis of the liver, Parkinson's disease, Huntington's disease, multiple sclerosis (MS), amyotrophic lateral sclerosis (ALS), osteoarthritis, rheumatoid arthritis or other form of arthritis, diabetes mellitus, emphysema, macular degeneration, or glomerulonephritis.

The step of assessing adaptability of the patient's perception of bodily self-image generally comprises a sensory-perceptual paradox, a computerized assessment tool, a virtual reality effect, an indicia of neurological activity, or an indicia of brain activity.

In one embodiment, the patient is preferably presented with a sensory-perceptual paradox comprising stimulation of the visual sense, a visual illusion, visual effects, or digital imagery. The sensory paradox involves at least one other sense, wherein the visual sense and other sense are each stimulated as part of the paradox. In one embodiment, the visual and at least one other sense are stimulated simultaneously or nearly simultaneously.

In certain embodiments, the sensory paradox is at least partially created using a digital medium or a computer processor. The at least one other sense is somatosensory or tactile, or in some embodiments, kinesthetic.

The likelihood that the patient will experience a response shift is a function of the time, duration, intensity and/or extent, or any combination thereof, of the candidate's response to the assessment, or a function of the degree of paradox presented to the patient.

As with other aspects of the invention disclosed herein, the patient's response to the assessment can be determined from an objective measure, such as a measure of neurological activity or brain activity, or an image thereof, or a measure of skin conductance.

In certain presently preferred embodiments, the objective measure is obtained via skin conductance resonance (SCR), electroencephalography (EEG), magnetic resonance imaging (MRI), computed tomography (CT), positron emission tomography (PET), electronystagmography (ENG), single photon emission computed tomography (SPECT) or by measuring evoked potentials.

In one embodiment, the sensory-perceptual paradox comprises stimulation of the visual sense by allowing the patient to view a simulated or artificial body or body part corresponding to an actual body or body part, and stimulation of the somatosensory sense by simultaneously touching the simulated or artificial body or body and the corresponding actual body or body part.

The patient's corresponding actual body part is preferably not in the patient's visual field while the simulated or artificial body or body part is visible to the patient.

As provided above, the degree of paradox is a function of how life-like the simulated or artificial body or body part is, wherein the more life-like the simulated body or body part is the lower the degree of paradox and the less life-like the simulated body part is, the higher the degree of paradox.

In yet another of the several aspects of the invention disclosed herein provided are methods of conducting a quality of life (QOL) study comprising the steps of:
providing a plurality of subjects for the study;
for each subject:
   providing over time a QOL assessment on each of a plurality, p, of occasions to obtain assessment data;
   determining a score or scores for each such QOL assessment;
   determining from the score or scores a baseline QOL response(s), based on the subject's score or scores for an initial number, n, of such occasions; such that p is much greater than n;
   monitoring the subject's score or scores for each subsequent QOL assessment for unexpected deviations from the baseline QOL response; wherein an unexpected deviation is defined as part of the study;
   ascertaining whether there are any known factors that may explain the unexpected deviation from the subject's baseline QOL response;
   if there are no ascertainable factors that explain the unexpected deviation from the subject's baseline QOL response, assessing the propensity of a subject to experience an improved QOL due to a response shift; and
   determining from the testing whether the subject shows a propensity to experience a response shift;
eliminating from the QOL study assessment data from subjects who are determined to show a propensity to experience an improved QOL due to a response shift;
completing the QOL study or any portion thereof, and analyzing the results thereof without the eliminated assessment data.

The skilled artisan will appreciate that such studies may be conducted over a long period of time, such as weeks, months, years and even decades. Thus “completing” for purposes here does require that every aspect of a study (e.g. the entire study) be completed, but rather that a portion thereof is completed such that sufficient assessments have been obtained to make analyzing the results useful, separately for a single subject, or for a plurality of the subjects in the study.

The skilled artisan will understand that the methods are generally intended to improve analysis of data from QOL, such as longitudinal studies for QOL taken over an extended period of time in persons, for example patients visiting a physician's office. The subjects for the methods may be ‘normal’ subjects with no particular medical conditions, or they may be people who share a particular condition or set of conditions, such as a health issue. Subjects may be chosen across a wide variety of traits, such as geographic, educational, or career background, health status, age, gender, or the like, or the subjects may be randomly or broadly accepted into the study.

With respect to the variables n and p, n is some fraction of p. The skilled artisan will appreciate that in order to have a statistically useful baseline or measure to which later results or scores can be compared, more than one assessment will be needed. In one embodiment, p is much greater than n. By “much greater” it is intended that p is at least 2 times greater than n. In other embodiments, p can be 3, 4, 5, 6, 7, 8, 9, or 10 greater than n, or even more. In preferred embodiments, n comprises a number that is less than about one-fifth or one tenth of the total number p of the plurality of assessments.

The skilled artisan will also understand that a degree of variation in the scores or responses to any such assessment is normal and thus expected. By “unexpected deviation” is intended that the amount of deviation is greater than any normal amount of deviation that might reasonably be anticipated among the assessment for that subject or for across all subjects. The skilled researcher will also understand how to determine or define such an “unexpected deviation” which could be based on any useful measure, for example as a percentage difference, or a certain number of standard deviations of difference between the baseline and the assessment score being compared.

The methods of assessing the propensity of a subject to experience an improved psychological condition due to a response shift are generally consistent with related methods disclosed hereinabove. The assessment preferably comprises presenting the subject with a sensory-perceptual paradox comprising stimulation of the visual sense, a visual illusion, visual effects, or digital imagery, wherein the sensory paradox involves at least one other sense, and wherein the visual sense and other sense are stimulated simultaneously or nearly simultaneously as part of the paradox. In one presently preferred embodiment, the other sense is somatosensory or tactile.

The likelihood that the subject will experience an improved psychological condition due to a response shift is a function of the time, duration, intensity and/or extent, or any combination thereof, of the subject's response to the assessment, or a function of the degree of paradox presented to the subject.

Preferably the subject's response to the assessment can be determined from an objective measure comprising a measure of neurological activity or brain activity, or an image thereof, or a measure of skin conductance.

In various embodiments the objective measure is obtained via skin conductance resonance (SCR), electroencephalography (EEG), magnetic resonance imaging (MRI), computed tomography (CT), positron emission tomography (PET), electronystagmography (ENG), single photon emission computed tomography (SPECT), magnetoencephalography (MEG), or superconducting quantum interference devices (SQUIDS), or by measuring evoked potentials.

The sensory-perceptual paradox comprises stimulation of the visual sense by allowing the subject to view a simulated or artificial body or body part corresponding to an actual body or body part, and stimulation of the somatosensory sense by simultaneously touching the simulated or artificial body or body part and the corresponding actual body or body part, wherein the corresponding actual body or body part is not in the subject's visual field while the simulated or artificial body or body part is visible.

In one embodiment, the degree of paradox is a function of how life-like the simulated or artificial body or body part is, wherein the more life-like the simulated body or body part is the lower the degree of paradox and the less life-like the simulated body or body part is, the higher the degree of paradox. In one embodiment, the degree of paradox relates to the positioning of the simulated or artificial body or body part, for example the angle relative to where the subject's corresponding actual body or body part would be located.

A further aspect of the invention provides methods of selecting a course of therapy for a patient suffering from a terminal, chronic, progressive, or degenerative disease or condition, the method comprising the steps of:
determining which courses of therapy provide an option that might produce a desirable outcome for the patient;
for each option, considering the likelihood that the course of therapy will extend the life of the patient, alleviate the suffering of the patient, or otherwise improve the patient's physical or psychological situation;
assessing the likelihood that the patient will experience an improved psychological condition due to a response shift;
determining the cost-effectiveness for each option;
considering any other factors relevant to the therapy or patient; and
selecting a course of therapy for the patient based on the cost-effectiveness, and the likelihood that the patient will experience a response shift; and optionally, the other factors.

The skilled artisan will appreciate that a strict or applicable standard of care for terminal patients and patients with chronic and degenerative disorders is very difficult to establish. The issue is on the cutting edge of medical ethics, and the fact of the matter is that cost-benefit or cost-effectiveness analyses are taken into consideration as a practical matter. The methods provided herein allow a care provider organization to consider an important factor that has not previously been available to them. In a population of response shifters, i.e. people who are presumably more readily able to shift into their “experiencing self” may tend to self report less pain, being more comfortable, having less stress and anxiety over their situation, and the like. Accordingly, a method that allows these people to be identified permits the care provider to determine a proper therapeutic treatment or course of treatment that may differ from a population of primarily people unlikely to experience a response shift, while maintaining the patient's comfort levels and the highest standards of medical ethics. Since such methods may be utilized not only by organizations providing therapeutic care, but perhaps by organizations providing palliative treatment or even hospice when there are no further “therapeutic” options, “course of therapy” as with respect to this aspect of the invention includes merely palliative treatment, e.g. treatment intended only to lessen pain. If a population of likely response shifters will tend to report less pain, then it follows that doses or medicines required to keep them comfortable may be less than those for nonresponse shifters. Using less medicine may also permit a safer course of therapy.

The methods preferably include a step of assessing the likelihood that the patient will experience an improved psychological condition due to a response shift which comprises presenting the patient with a sensory-perceptual paradox comprising stimulation of the visual sense, a visual illusion, visual effects, or digital imagery. The sensory paradox in one embodiment involves at least one other sense, wherein the visual sense and other sense are stimulated simultaneously or nearly simultaneously as part of the paradox. Preferably the at least one other sense is somatosensory or tactile.

The likelihood that the patient will experience an improved psychological condition due to a response shift is a function of the time, duration, intensity and/or extent, or any combination thereof, of the candidate's response to the assessment, or a function of the degree of paradox presented to the patient.

The patient's response to the assessment can be determined from an objective measure comprising a measure of neurological activity or brain activity, or an image thereof, or a measure of skin conductance.

Examples of suitable objective measures include those obtained via skin conductance resonance (SCR), electroencephalography (EEG), magnetic resonance imaging (MRI), computed tomography (CT), positron emission tomography (PET), electronystagmography (ENG), single photon emission computed tomography (SPECT) or by measuring evoked potentials.

In one embodiment, the sensory-perceptual paradox comprises stimulation of the visual sense by allowing the person to view a simulated or artificial body or body part corresponding to an actual body or body part, and stimulation of the somatosensory sense by simultaneously touching the simulated or artificial body or body and the corresponding actual body part, wherein the corresponding actual body or body part is not in the patient's visual field while the simulated or artificial body or body part is visible.

The degree of paradox is a function of how life-like the simulated or artificial body or body part is, wherein the more life-like the simulated body or body part is the lower the degree of paradox and the less life-like the simulated body or body part is, the higher the degree of paradox.

In another aspect, the invention provides methods for predicting that an individual is likely to be a placebo responder or is likely to experience a response shift. The method comprises the steps of
obtaining an objective measure of brain activity in the individual, and
determining therefrom whether the individual is likely to be a placebo responder or is likely to experience a response shift, wherein the measure of brain activity is correlated with performance in an assessment of adaptability of perception of bodily self-image.

As with the foregoing aspects, the objective measure is obtained via skin conductance resonance (SCR), electroencephalography (EEG), magnetic resonance imaging (MRI), computed tomography (CT), positron emission tomography (PET), electronystagmography (ENG), single photon emission computed tomography (SPECT) or by measuring evoked potentials.

In certain presently preferred embodiments, the objective measure is obtained via functional MRI (fMRI) or Quantitative EEG (QEEG).

In a final aspect, the invention provides a database comprising a collection of data useful for establishing a correlation between the objective measure of brain activity and an assessment of adaptability of perception of bodily self-image.

### REFERENCES

A. Harrington, Ed., (1997). The Placebo Effect: An Interdisciplinary Exploration, Harvard: Cambridge.
Armel, K. C. & Ramachandran, V. S. (2003). Projecting sensations to external objects: Evidence from skin conductance response. Proceedings of the Royal Society of London: Biological, 270, 1499-1506.
Botvinick, M. & Cohen, J. D. (1998). Rubber hand ‘feels’ what eyes see. Nature, 391, 756.
Guterstam, Petkova, and Ehrsson ‘The Illusion of Owning a Third Arm’ PLoS ONE 6(2): e17208 (2011).
Kahneman and Riis, (2005). Living, and thinking about it: Two perspectives on life. In F. A. Huppert, N. Baylis & B. Keverne (Eds.), The science of well-being (pp. 285-304). Oxford: Oxford University Press.
Petkova V I, Ehrsson H H (2008) If I Were You: Perceptual Illusion of Body Swapping. PLoS ONE 3(12): e3832. doi:10.1371/journal.pone.0003832
Sprangers, M A & Schwartz, C E, (1999) Integrating response shift into health-related quality of life research: a theoretical model. Soc. Sci. Med 48:1507-1515.
van der Hoort, Guterstam, and Ehrsson (2011) ‘Being Barbie: The Size of One's Own Body Determines the Perceived Size of the World’ PLoS ONE 6(5): e20195.
Wilson, I B (1999). Clinical understanding and clinical implications of response shift: Societies & health in transition. International Conference on the Social Sciences & Medicine No. 15, Antalya, TURQUIE, 48(11):1507-1588 (2 p.):1577-1588.

### EXAMPLES

The invention can be further illustrated by the following examples, although it will be understood that the examples are included merely for purposes of illustration and are not intended to, and do not limit the scope of the invention unless otherwise specifically indicated.

### Example 1

### Methods of Presenting a Sensory-Perceptual Paradox

For all of the hypothetical examples which follow, the method of assessing the subject's ability to shift their perception of bodily self-image can be measured as follows (including many variations thereof):

A subject is presented with a sensory perceptual paradox that is generated purely physically (e.g. the rubber arm illusion or similar presentation), partly physically and partly with digital imagery or computer technology or the like (such as the mannequin body paradox described above using a helmet and camera), or purely via digital, computer, or other technological means (for example using virtual reality).

The subject is presented with the paradox under conditions which involve at least two senses, most preferably sight and touch. The visual sense is preferred for use herein although other assessments using other senses may be developed based on the disclosure herein.

The subject may be presented with varying degrees of paradox, using for example, simulated or artificial body or body parts that vary in their actual appearance from very lifelike, to completely not lifelike (e.g. a wooden block), or by using positioning of the paradox e.g. at increasingly less probably angles or distances. For example a simulated hand that is immediately adjacent to the subject's actual hand present less paradox than a simulated hand that is one foot, two feet, or even three feet or more away from the subject expects their own hand to be located in space. The paradox may become effective by presenting different scenarios to the subject. For example, in one paradox, a simulated body part may be touched lightly with a feather, while in another, the body part may be threatened to various degrees, for example with a blow (e.g. a doctor's reflex hammer versus a carpenter's hammer) or a sharp instrument (e.g. a pin versus a knife or blade). The paradox may also present other “experiences” such as electrical stimulation, vibration, heat, cold, or other kinesthetic stimulation—all of which might be used to vary the degree of paradox for the subject.

The subject's ability to experience a shift on the perception of their bodily self-image can be measured by subjective means (e.g. asking the subject about their experiences or self-reporting) or by one or more objective measures that reflect the shift. Here, as in all examples below, the measurements (and criteria based thereon) can be indicative of the time required to experience a shift, or alternatively, the extent of the shift experienced. Correlations between the objective measure and the subjective means may be established to assist with the interpretation of the objective measures, and thereafter a database can created and used to help interpret the measurements obtained objectively.

### Example 2

### A-Priori Prediction of Placebo Effect (Fixed Subject Pool)

In this hypothetical example, immediately prior to the start of a 42-day clinical trial involving the treatment of anxiety in patients over the age of 18 years of age, each of the prospective subjects will be assessed via the method described in Example 1 prior to any subject being formally initiated into the study. Those subjects determined to be in the top 22% of the pool of subjects (e.g. the fastest 22% based on the time to switch perception of bodily self-image, or alternatively those 22% with the greatest extent of shift in perception of bodily self-image) are defined as placebo responders and eliminated from the study a-priori. The number of subjects in the pool is such that after the elimination of the 22% fastest responders (or alternatively the 22% of subjects who shifted their body self-image most significantly), the number of remaining subjects is sufficient to allow the study to be conducted and completed with adequate numbers.

### Example 3

### A-Priori Prediction of Response Shift (Fixed Subject Pool)

In this hypothetical example, immediately prior to the start of a 30-day clinical trial involving the treatment of pain in patients over the age of 18 years of age, all prospective subjects will be assessed via the method provided in Example 1 prior to any subject being formally initiated into the study. Those subjects in the top 20% (the fastest time to switch) will be response shifters to questions requiring answers in self-reported form. Those subjects can be eliminated from the trial a priori.

### Example 4

### A Priori Prediction of Response Shift (Rolling Subject Pool)

In this hypothetical example, immediately prior to the formal start of a 30-day clinical trial involving the treatment of angina in patients over the age of 18 years of age, each prospective subject will be assessed via the method described above as they become otherwise eligible to participate (e.g. after they have satisfied other criteria for inclusion and/or exclusion). A normative database will have been created and the results of each subject can readily be compared to the database. Those subjects in the top 17.5% (the fastest time to switch) of the normative database will be response shifters to questions requiring answers in self-report form. These subjects may be withdrawn from the study based on this a priori identification.

### Example 5

### A Priori Prediction of Placebo Response (Rolling Subject Pool)

In this hypothetical example, immediately prior to the formal start of a 30-day clinical trial involving the treatment of gastric ulcer patients over the age of 18 years of age, each subject will be assessed via the method as they become eligible to participate. A normative database will have been created and the results of each subject can be compared to the database. Those in the top 21% (e.g. the fastest time to switch, or the greatest extent of switch) of the normative database will be placebo responders. These subjects may be withdrawn from the study based on this a priori identification.

### Example 6

### Retrospective Validation of Response Shift (Fixed Subject Pool)

In this hypothetical example, subsequent to a completed clinical trial in depression, subjects who have been classified as possible response shifters and not response shifters will be identified. That classification will be kept confidential until the completion of a retrospective validation of the classification. In the retrospective validation study, all subjects in the completed clinical trial for depression will be assessed by the method. Those in the top 12% (based e.g. on the fastest time or greatest extent) will be deemed/confirmed response shifters and this identification will confirm any previous classification as such. For those subjects who are confirmed response shifters, the data may be safely eliminated from the raw study results, or the data for those subjects may be statistically adjusted or weighed to account for the effect of any observed response shift in the data.

### Example 7

### Retrospective Validation of Placebo Effect (Fixed Subject Pool)

In this hypothetical example, subsequent to a completed clinical trial in asthma, subjects who have been classified as placebo responders and not placebo responders will be identified. That classification will be kept confidential until the completion of a retrospective validation of the classification. In the retrospective validation study, all subjects in the completed clinical trial for asthma will be assessed by the method of Example 1. Those subjects in the top (the fastest time to switch) 12% will be deemed/confirmed placebo responders and this identification will confirm any previous classification as placebo responders. For those subjects who are confirmed placebo responders, their data may be safely eliminated from the raw study results, or the data for those subjects may be statistically adjusted or weighed to account for the effect of any observed placebo effect in the data.

### Example 8

### A Priori Prediction of Successful Pharmacotherapy

In this hypothetical example, prior to initiation of drug therapy for chronic fatigue, a patient will be assessed by the method of Example 1. Using a normative database of response times across a population of people, the physician will expect a higher probability of a patient's self report of successful treatment if the prospective patient is in the top 50% (fastest) of time to switch bodily self-image. Criteria based on extent of shift in perception can also be used instead of time to switch.

### Example 9

### A-Priori Prediction of Successful Psychotherapy

In this hypothetical example, prior to initiation of psychotherapy for social anxiety, a prospective patient will be assessed by a method according to Example 1. Using a normative database of response times across people, the psychologist will expect a higher probability of a patient's report of a successful outcome of psychotherapy if the prospective patient is in the top 33% (fastest) of time to switch the perception of bodily self-image.

### Example 10

### Assessment of the Degree of Acceptance of Palliative Care at the End-of-Life

In this hypothetical example, in the consideration of acceptance of palliative care at the end of life, a person will be assessed by one of the methods described in Example 1. Using a then existing normative database of response times across a plurality of people, the care team will expect higher self reported acceptance of palliative care if the person is in the top 15% (fastest) of time to switch to bodily self-image.

### Example 11

### Assessment of the Potential for a Person to Achieve Benefit from Placebo Treatment, Standard Quality of Life, Anxiety, Depression, Pain, or the Like

In this hypothetical example, standard scale(s) for assessing one or more the above conditions (e.g., EuroQOL, Hamilton Anxiety scale, Zung Depression Scale, Numeric Rating Scale) will be administered to a subject at time t1. Subsequently, the subjects will be assessed by a variation on the method disclosed in Example 1. When fully involved in the sensory perceptual paradox, they will again be asked questions from the standard scales (i.e. at time t2). The difference(s) between their scores on the standard scales from t1 to t2 will indicate whether or not they can achieve benefit from placebo treatment. People with greater than a 10% improvement in scores will benefit from an administered placebo.

### Example 12

### A Clinical Protocol for Evaluating Whether Changes in Perceived Identity Alter Self-Reporting of Anxiety, Depression, Pain or Quality of Life

Study Hypothesis

The study hypothesis is that perceptions about one's identity influences feelings of distress and quality of life. Specifically, it is hypothesized that when subjects report their identity as being separated from their physical body (i.e., when the experiencing self is dominant), their scores on the health or QOL assessments will show changes towards improvements. It is also hypothesized that greater score improvements enhance credibility for the theory that shifts of identity, between the experiencing self and the remembering self, are the cause of placebo response and response shift.

Methods

Participants

Patients with diagnoses of mild to moderate anxiety, depression and pain will be studied, as will normal, healthy subjects who will be assessed for their quality of life. All subjects will be adults between the ages of 18 and 65 years of age.

The sample sizes have been chosen based on effect sizes seen in the Petkova and Ehrsson report. With 10-20 subjects their experiments, statistical significance in their findings appeared at the p<0.002 level. There is no reason to believe effect sizes in the current studies should be different from what might be expected in this research since the experimental procedure and the type of questions asked will be very similar to those used by Petkova and Ehrsson.

The time commitment of a subject will be 7 days (+/−1). Subjects must attend the research clinic on study days one and seven and complete all assessments on those days. Subjects will be considered to have successfully completed the experiment if they complete all assessments on each of the two required days.

In experiment 1, an assessment of the influence of changes in perceived self-identity on quality of life, 40 adult, study-naïve healthy volunteers will participate.

In experiment 2, an assessment of the influence of changes in perceived self-identity on depression, 40 adult, study-naïve subjects with a history of mild to moderate depression will participate.

In experiment 3, an assessment of the influence of changes in perceived self-identity on anxiety, 40 adult, study-naïve subjects with a history of mild to moderate anxiety will participate.

In experiment 4, an assessment of the influence of changes in perceived self-identity on pain, 40 adult, study-naïve subjects with a history of mild to moderate chronic neuropathic pain will participate.

All participants will give written informed consent prior to participating in the relevant experiment. Subjects will receive an honorarium for their participation. The local Ethical Committee will approve this research prior to its conduct.

Randomization

Two randomizations are required. Each subject will be randomized for:

1. Assignment for the study day on which they will receive the active intervention (1 vs. 7); and

2. For the symptom recall period to be used (“now” vs. in the last 30 days).

Symptom Recall Period

The time period for the patient reports is an important variable that must be especially well controlled in this study because the reporting from either the experiencing self or the remembering self depends on a time component. The immediate present is the domain of the experiencing self whereas cognitive recollection (memory) of the past, no matter how recent, is the domain of the remembering self.

Because the intervention using the head-mounted visual display theoretically encourages subjects to respond from the perspective of the experiencing self, the assessments for a randomized half of all subjects will utilize the symptom recall period of the present moment (i.e., “right now”). This will establish the pure effect of the HMD intervention. It compares the “right now” condition of both the experiencing self and the remembering self—even though the remembering self theoretically cannot provide an answer for the immediate moment.

However, the maximal effect of the intervention is calculated by contrasting responses from the remembering self (i.e., using a recall period of “in the last 30 days”) against that from the experiencing self. To achieve this, half of all subjects will be asked to respond using the recall period of the last 30 days for all assessments—even though the experiencing self cannot theoretically answer questions requiring such memory. Although this symptom recall period encourages responses from the remembering self, the effect of the intervention should encourage the experiencing self to emerge despite the instructions.

Health and QOL Assessments

The assessments chosen here are those classically used by the pharmaceutical industry for use in clinical trials for NDA submissions. Alternative assessments, probably with dramatically improved sensitivity, can be also done using, for example, the NIH's PROMIS program. A full discussion of the pros and cons of such approaches is beyond the scope of this disclosure. For the interested reader, more information about PROMIS can be found at http://www.nihpromis.org/about/overview.

Subjects in Experiment 1 will be assessed with the use of the SF-36 and the Health Utility Index, Mark III (HUI-3). Subjects in Experiment 2 will use the Hamilton Depression Scale (HAM-D) and the HUI-3. Subjects in Experiment 3 will use the Hamilton Anxiety Scale (HAM-A) and the HUI-3. Subjects in Experiment 4 will use Jensen's (2008) neuropathic pain scale and the HUI-3. The HUI-III has been a favorite of quality of life researchers for more than 20 years since it is easy to administer, has successfully passed tests of validity and the results can be statistically transformed into health utilities The HAM-A and HAM-D assessments are the approved, standard rating instruments most frequently used in the clinical trials of new anxiolytic and antidepressant drugs submitted to the US FDA for regulatory approval. Jensen's pain assessment measure is in keeping with 2005 and 2008 Initiative on Methods, Measurement, and Pain Assessment in Clinical Trials (IMMPACT) recommendations.

Collection of Subject's Responses

All health-related assessments used here are self-reported and typically answered manually using paper and pencil or a computer keyboard. However, for these experiments it is not feasible for subjects to manually answer questions because of the head-mounted display. It obstructs or alters their view and is part of the equipment used to separate their hand from their identity. Given these issues, all subject responses will be oral. The experimenter will ask the questions and record their answers. Subjects will review the recorded responses once that day's experiment has been completed to ensure the recording has been accurate. Answers may only be changed (only by the recorder) if the subject indicates a mistake was made between what was initially said and what was initially recorded.

Questionnaire evidence for perceiving a mannequin's body as one's own will be collected using the 7-item questionnaire of Petkova and Ehrsson of a similar questionnaire. The Petkova and Ehrsson questionnaire consists of the seven statements related to an alignment of the subject's identity to that of the mannequin (e.g., “The mannequin's body began to resemble my own body in terms of shape, skin tone, or some other visual feature”). Subjects will offer their answers orally and immediately after the intervention has been completed (at the conclusion of the stroking) according to a seven-point scale ranging from ‘agree strongly’ (+3) to ‘disagree strongly’ (−3).

Skin Conductance Recording (SCR)

To confirm that subjects actually experience perceived separation from their physical body, skin conductance will also be recorded. Skin conductance electrodes will be placed on the subject's left wrist immediately prior to donning the HMD.

SCR will be used as the measure of autonomic nervous system arousal because it is not easily prone to movement artifacts and is a good predictor of psychological arousal. Normal subjects cannot voluntarily control their SCRs, thus unlike self-reports of emotional arousal, SCR results cannot be ‘faked’ or be the result of task demands. The subject's perceptual point of view will be assessed through their seeing the abdominal “cutting” of the mannequin and the recording of SCR in response to that sight.

SCR will be recorded with Ag—AgCl electrodes from the thenar and hypothenar eminences of the left hand. Data will be recorded through an acquisition unit and related software (e.g., Biopac MP100 with AcqKnowledge v. 3.4.1 software). SCRs will be quantified in the following manner: the amplitude of the largest SCR greater than 0.03 microsiemens that occur 1-5 s from the beginning of simulated “cutting” of the mannequin's torso will be scored as a response to that stimulus. Following standards, SCR magnitudes will be recorded, meaning that SCR amplitudes of zero will be included in analyses. Subjects who exhibit SCR magnitudes of zero to all stimuli will be classified as SCR non-responders and excluded from analyses.

The parameters of the recording will be as follows: The gain switch will be set to 5 mho/V and the CAL2 Scale Value will be set to 5 (middle). The timing of the threat events will be indicated in the raw data files by the experimenter pressing a switch button during the SCR recordings.

Intervention—Head Mounted Display (HMD) and Human Mannequin

The experimental condition's intervention consists of a subject wearing of a head-mounted display, feeling stroking on their abdomen and seeing the simultaneous stroking and mock “cutting” of a mannequin's abdomen thorough the helmet's video display. The control condition's intervention consists of a subject wearing a head-mounted display, feeling stroking on their abdomen but not seeing viewing the simultaneous stroking or mock “cutting” of a mannequin's abdomen thorough the helmet's video display (because it has been turned off). The subject in the control condition will only see a video display illuminated by a soft white light.

The head mounted display (HMD) (e.g., Cybermind Visette Pro PAL, Cybermind Interactive, Maastricht, the Netherlands; Display Resolution=6406480; true stereoscopic vision, with a wide field-of-view (diagonal field of view=71.5 u) will be connected to two synchronized color CCTV cameras (e.g., Protos IV, Vista, Wokingham, Berkshire, UK) attached side-by-side to special helmets. The spacing between the cameras will be adjusted for each participant to ensure it matches the inter-pupillary distance between their eyes (typically 8-10 cm). The CCTV signals will be relayed directly to the HMDs, without any software conversion, and thus will be presented without noticeable delay. The cameras will be attached to a helmet affixed to the head of an unclothed, full sized, human mannequin and will be aimed downwards so the HMD shows the torso, legs and feet of the mannequin.

Experimental Procedure

During the clinic visit at days 1 and 7, and with the assistance of the investigator, subjects will place the SCR leads on their left wrists and head mounted display helmet on their head and undergo four one minute long periods of synchronous stroking (each stroke will be approximately 3 cm long; about 60 strokes will be applied per minute) along the midline of their abdominal area. This will occur in synchrony with stroking of the abdominal area of the mannequin. Immediately at the end of the period of synchronous stroking, but while still in the HMD, subjects will complete all health-related assessments. Next, they will be asked about the location of their identity (i.e., using the 7 item scale) and then through their HMD, subjects in the experimental condition will see the experimenter horizontally “cut” the torso of the mannequin with a knife. Changes in SCR will affirm or deny whether a shift in identity has taken place (i.e., increased skin conductance will affirm the subject perceives a real threat to themselves and that therefore their identity has aligned with that of the mannequin). Subjects in the control condition will not see the experimenter horizontally drag a knife across the torso of the mannequin. The study concludes for each patient after his or her SCR has been successfully obtained.

Questionnaire for Perceiving a Mannequin's Body as One's Own. (Petkova and Ehrsson, 2008)

1. I seemed to feel the touch given to the mannequin.

2. It seemed as though the touch I felt was caused by the stick touching the mannequin's body.

3. I felt like the mannequin's body was my body.

4. I felt naked.

5. I felt as if I had two bodies.

6. I felt as if my body had turned into a plastic body.

7. The mannequin's body began to resemble my own body in terms of shape, skin tone, or some other visual feature.

Subjects will answer these questions using a seven-point response scale ranging from ‘agree strongly’ (+3) to ‘disagree strongly’ (−3).

### Example 13

### Assessment of the Extent of a Individual's Shift in Perception of Bodily Self-Image

In this hypothetical example, standard scale(s) for assessing e.g. health status, quality of life, or the like will be administered to a subject. Subsequently (e.g. at a later time or date), the subjects will be presented with a sensor-perceptual paradox according to Example 1 or a variation thereof. The subjects will experience the paradox under tightly standardized conditions—subjects will be presented with the identical sensory perceptual paradox, the same instructions before during and after the paradox is presented, and will have the paradox presented with standardized timing as to both the presentation and the duration of the paradox. Upon achieving a shift in perception of bodily self-image, or after a standardized amount of time experiencing the paradox, the subjects will again be asked questions from the standard scale. Individuals who demonstrate greater improvement in their own scores after experiencing the paradox, making the shift, will be those who are more likely to be placebo responders or response shifters. For example hypothetical Subject A scores 50% on a standardized QOL assessment prior to experiencing the paradox, and her score improves to 90% after experiencing the paradox. Subject B also scores 50% pre-paradox, but only scores 70% after experiencing the sensory-perceptual paradox. Subject A can be said to be have shifted their perception to a greater extent than Subject B, and Subject A will more likely to be a placebo responders and/or a response shifter than Subject A. Under appropriate circumstance, this information can be used to eliminate Subject A a priori from a clinical study, or to tailor treatments for certain conditions for Subject A. The comparison among various individuals (and the determination of e.g. which subjects are the most likely placebo responders) in such tests could be on raw score differences or on any other basis related to those scores (e.g. subjects with the highest x % of score differences, or those subject who are 2 standard deviations above average, etc).

The disclosure and foregoing examples explore radical findings with potentially broad implications and practical implications related to health, healthcare, and treatment. Fundamental questions arise about whether people can be reliable and valid reporters of health. There are significant implications for in the field of subjective valuations of health states, health economics and treatments across various sectors of the population. Moreover, there are implications for response shift, quality of life reporting and variability in the responses to clinical and outcome measures. Finally, because separation of the body and identity can occur, there are also significant implications for understanding the cause of placebo responding and response shift.

The scope of the invention is set forth in the claims appended hereto, subject, for example, to the limits of language. Although specific terms are employed to describe the invention, those terms are used in a generic and descriptive sense and not for purposes of limitation. Moreover, while certain presently preferred embodiments of the claimed invention have been described herein, those skilled in the art will appreciate that such embodiments are provided by way of example only. In view of the teachings provided herein, certain variations, modifications, and substitutions will occur to those skilled in the art. It is therefore to be understood that the invention may be practiced otherwise than as specifically described, and such ways of practicing the invention are either within the scope of the claims, or equivalent to that which is claimed, and do not depart from the scope and spirit of the invention as claimed.

What is claimed is:

1. A method of selecting participants for a biomedical or health-related research study (“clinical trial”) comprising the steps of:
(a) establishing at least one inclusion and/or exclusion criterion for the study that encompasses a measure of a prospective participant's propensity to respond to placebo treatment;
(b) eliminating, a priori, from the study any prospective participant who does not meet the required criteria for inclusion or exclusion;
wherein the measure of propensity to respond to placebo treatment comprises an assessment of the adaptability of the prospective participant's perception of their bodily self-image.

2. The method of claim 1 wherein:
(a) an exclusion criterion excludes prospective participants who have the ability, in a specified assessment, to shift their perception of their bodily self-image:
(i) within specified time(s);
(ii) within relative time(s) based on the times for all prospective participants for the study;
(iii) within time(s), percentage(s) or other measure(s) determined from a database comprising assessment data related to adaptability of the perception of bodily self-image from a plurality of people; and/or
(iv) to a specific extent, or within a range of extents as compared to all prospective participants for the study, or as determined from a database comprising assessment data related to adaptability of the perception of bodily self-image from a plurality of people; or
(b) an inclusion criterion requires that, in a specified assessment, prospective participants do not shift their perception of their bodily self-image
(i) faster than a specified time;
(ii) faster than a relative time based on the times for all prospective participants for the study;
(iii) faster than a defined time, or within a percentage, or other measure determined from a database comprising assessment data related to adaptability of the perception of bodily self-image from a plurality of people; and/or
(iv) to a specific extent, or within a range of extents as compared to all prospective participants for the study, or as determined from a database comprising assessment data related to adaptability of the perception of bodily self-image from a plurality of people.

3. The method of claim 1, wherein the assessment of adaptability of the perception of bodily self-image for a prospective participant comprises a sensory-perceptual paradox, a computerized assessment tool, a virtual reality effect, an indicia of neurological activity, or an indicia of brain activity.

4. The method of claim 3 wherein the sensory-perceptual paradox is at least partially created using electronic equipment, a computer processor, or a digital medium.

5. The method of claim 3 wherein the sensory-perceptual paradox comprises stimulation of the visual sense, a visual illusion, visual effects, or digital imagery.

6. The method of claim 5 wherein the sensory-perceptual paradox involves at least one other sense, wherein the visual sense and other sense are simultaneously or nearly simultaneously stimulated as part of the paradox.

7. The method of claim 6 wherein the at least one other sense is somatosensory or tactile.

8. The method of claim 7 wherein the sensory-perceptual paradox comprises a simulated or artificial body or body part; stimulation of the visual sense comprises allowing the prospective participant to view the simulated or artificial body or body part; and stimulation of the somatosensory sense comprises simultaneously touching the simulated or artificial body or body part and the corresponding actual body or body part of the prospective participant.

9. The method of claim 8 wherein the simulated or artificial body or body part:
a) is lifelike and presents a less extreme paradox to the prospective participant's perception; or
b) is not lifelike and presents a more extreme paradox to the prospective participant's perception.

10. A method for determining the likelihood that a candidate for a biomedical or health-related research study (“clinical trial”) will respond to a placebo used in the clinical trial, the method comprising the steps of assessing adaptability of the candidate's perception of bodily self image; and determining a likelihood that the candidate will respond favorably to a placebo based on the candidate's response to the assessment.

11. The method of claim 10 wherein the candidate is otherwise qualified to be a participant in the clinical trial based on the inclusion and exclusion criteria for the clinical trial.

12. The method of claim 10 wherein likelihood of being a placebo responder can be used as an additional criterion for inclusion in or exclusion from the study.

13. The method of claim 10, wherein the step of assessing the adaptability of the candidate's perception of bodily-self image comprises a sensory-perceptual paradox, a computerized assessment tool, a virtual reality effect, an indicia of neurological activity, or an indicia of brain activity.

14. The method of claim 13 wherein the sensory-perceptual paradox comprises stimulation of the visual sense, a visual illusion, visual effects, or digital imagery.

15. The method of claim 14 wherein the sensory paradox is at least partially created using a digital medium or a computer processor.

16. The method of claim 14 wherein the sensory paradox involves at least one other sense, wherein the visual sense and other sense are each stimulated as part of the paradox.

17. The method of claim 16 wherein the visual and other sense are stimulated simultaneously or nearly simultaneously.

18. The method of claim 16 wherein the at least one other sense is somatosensory or tactile.

19. The method of claim 10 wherein the likelihood that the candidate will respond to a placebo is a function of the time, duration, intensity and/or extent or any combination thereof, of the candidate's response to the assessment.

20. The method of claim 10 wherein the candidate's response to the assessment can be determined from an objective measure.

21. The method of claim 20 wherein the objective measure comprises a measure of neurological activity or brain activity, or an image thereof, or a measure of skin conductance.

22. The method of claim 20 wherein the objective measure is obtained via skin conductance resonance (SCR), electroencephalography (EEG), magnetic resonance imaging (MRI), computed tomography (CT), positron emission tomography (PET), electronystagmography (ENG), single photon emission computed tomography (SPECT) or by measuring evoked potentials.

23. A collection of data comprising, for each of a plurality of people, data based on or obtained during an assessment of the adaptability of the person's perception of their bodily self-image.

24. The collection of data of claim 23 wherein the assessment of adaptability of the person's perception of their bodily self-image comprises a sensory-perceptual paradox, a computerized assessment tool, a virtual reality effect, an indicia of neurological activity, or an indicia of brain activity.

25. The collection of data of claim 24 wherein the data comprise one or more of the time, intensity, duration, or any combination thereof, of the candidate's response to the assessment.

26. The collection of data of claim 24 wherein the data comprise an objective measure of neurological activity, brain activity, or skin conductance related to an assessment of a person's perception of their bodily self-image.

27. A computerized system comprising:
a collection of data comprising, for each of a plurality of people, data based on or obtained during an assessment of the adaptability of the person's perception of their bodily self-image,
one or more data structures for arranging the data,
one or more data storage devices for storing the data,
a database management system adapted for managing the collection of data, and
one or more computers, servers, terminals, or networks for accessing the database management system, data structures, or data,
wherein the data are arranged according to the one or more data structures and stored at least temporarily on one or more data storage devices; and wherein the database management system, and one or more of the computers, servers, terminals, or networks are in data communication with each other and/or the data.

28. The computerized system of claim 27 further comprising a set of inclusion or exclusion criteria for a clinical trial, at least one criterion therein based on the collection of data.

29. A method of measuring a person's propensity to respond to administration of a placebo during a clinical trial comprising the steps of measuring (a) the time required for the person to experience a shift in perception of bodily self-image in response to a sensory-perceptual paradox comprising a visual paradox and stimulation at least one other sense, and/or (b) the extent of such a shift; and establishing that the time to shift and/or the extent of the shift are indicative of propensity to respond to the placebo.

30. The method of claim 29 wherein the other sense is somatosensory or tactile.

31. The method of claim 30 wherein the sensory-perceptual paradox comprises simultaneously or nearly simultaneously stimulating the person's visual and somatosensory senses.

32. The method of claim 29 wherein the propensity to respond to placebo administration is inversely related to the time required to shift perception of bodily self-image.

33. The method of claim 30 wherein the sensory-perceptual paradox comprises a simulated or artificial body or body part corresponding to an actual part of the person's body, stimulation of the visual sense comprises allowing the person to view the simulated or artificial body or body part, and stimulation of the somatosensory sense comprises simultaneously touching the simulated or artificial body and the corresponding actual body part.

34. The method of claim 33 wherein the actual part of the person's body is not in the person's visual field while the simulated or artificial body or body part is visible to the person.

35. The method of claim 29 wherein a shift in perception of bodily self-image is determined by a psychological measure or a physiological measure.

36. The method of claim 29 wherein a shift in perception of bodily self-image is determined subjectively by oral self-reporting by the person, or by answers to a paper and pencil assessment of the paradox.

37. The method of claim 29 wherein a shift in perception of bodily self-image is determined by an objective measure of neurological activity, brain activity, or skin conductance.

38. The method of claim 37 wherein the objective measure is obtained via skin conductance resonance (SCR) or an equivalent thereof, electroencephalography (EEG), magnetic resonance imaging (MRI), computed tomography (CT), positron emission tomography (PET), electronystagmography (ENG), single photon emission computed tomography (SPECT) or by measuring evoked potentials.

39. A method of improving data analysis for data from a clinical trial for a therapeutic treatment comprising the steps of:
(a) obtaining a set of raw clinical data;
(b) evaluating the raw clinical data by standard methods to generate preliminary results;
(c) obtaining the identity for each participant in the trial (i.e. unblinding the data);
(d) assessing the adaptability each participant's perception of their bodily self-image;
(e) determining which participants have readily adaptable body images;
(f) creating a modified clinical data set by modifying the raw clinical data to identify, eliminate, or statistically adjust data pertaining to those participants determined to have readily adaptable perceptions of their bodily self-images;
(g) evaluating the modified clinical data to generate modified results; and optionally,
(h) using the modified data or modified results in connection with seeking approval for the therapeutic treatment from a regulatory agency.

40. The method of claim 39 further comprising the step of comparing the preliminary results and the modified results to generate a comparison, and optionally using the comparison in connection with seeking approval from a regulatory agency.

41. The method of claim 39 wherein the steps of assessing the adaptability each participant's perception of bodily self-image and determining which participants have readily adaptable perceptions of bodily self-image comprise one or more of a sensory-perceptual paradox involving at least two senses, a computerized assessment tool, a virtual reality effect, a simulated or artificial body or body part; or a psychological or physiological measure of a shift in perception of bodily self-image perception.

42. A method of identifying subjects for a therapeutic treatment based on their propensity to respond favorably to a placebo treatment, the method comprising the step of measuring the ease with which the subject can experience a shift in their perception of bodily self-image; wherein the more easily a subject can shift their bodily self-image, the better subject they will be for the therapeutic treatment.

43. The method of claim 42 wherein the ease with which a subject can experience a shift in the perception of bodily self-image is a function of the time required for the subject to experience a given shift, the duration of the shift experienced, or the intensity or extent of the shift experienced.

44. The method of claim 42 wherein the subject is presented with a sensory-perceptual paradox, and the ease with which the subject can experience a shift in the perception of bodily self-image is a function of the degree of paradox presented to the subject.

45. The method of claim 44 wherein the sensory-perceptual paradox comprises stimulation of the visual sense by allowing the subject to view a simulated or artificial body or body part, and stimulation of the somatosensory by simultaneously touching the simulated or artificial body and the corresponding actual body part of the subject.

46. The method of claim 45 wherein the subject's corresponding actual body part is not in the subject's visual field while the simulated or artificial body or body part is visible to the subject.

47. The method of claim 45 wherein the degree of paradox is a function of how life-like the simulated or artificial body or body part is, wherein the more life-like the simulated body or body part is the lower the degree of paradox and the less life-like the simulated body or body part is, the higher the degree of paradox.

48. The method of claim 42 wherein the therapeutic treatment comprises a modified or reduced dosing regimen, a modified or reduced time of therapeutic treatment, a therapeutic treatment with fewer side effects than a standard of care therapy, an alternative to a standard of care therapy, or a placebo.

49. The method of claim 42 wherein the subject is suffering from a health-related condition comprising anxiety, an anxiety-related disorder, depression, a depression-related disorder, a neuropathy, or chronic pain, and wherein the therapeutic treatment is for treating said condition.

50. A method of determining, in patients with declining health, a propensity to experience a response shift, the method comprising the steps of assessing adaptability of the patient's perception of bodily self-image; and determining the candidate's propensity to experience a response shift, based on the assessment.

51. The method of claim 50 wherein the patient suffers from one or more of anxiety, depression, chronic pain, progressive degeneration of any physical or mental function, or low perceived quality of life.

52. The method of claim 50 wherein the patient is suffering from a terminal, chronic, progressive, or degenerative disease or condition.

53. The method of claim 52 wherein the disease or condition causes an impairment or loss of function of the central nervous system, peripheral nervous system, brain, heart, lungs, circulatory system, bones, joints, pancreas, kidneys, immune system, or any combination thereof.

54. The method of claim 52 wherein the patient has a terminal cancer, a neurodegenerative condition, a spinocerebellar ataxia, an encephalopathy, or other condition causing cerebellar degeneration, congestive heart failure, a muscular dystrophy, cirrhosis of the liver, Parkinson's disease, Huntington's disease, multiple sclerosis (MS), amyotrophic lateral sclerosis (ALS), osteoarthritis, rheumatoid arthritis or other form of arthritis, diabetes mellitus, emphysema, macular degeneration, or glomerulonephritis.

55. The method of claim 50 wherein the step of assessing adaptability of the patient's perception of bodily self-image comprises a sensory-perceptual paradox, a computerized assessment tool, a virtual reality effect, an indicia of neurological activity, or an indicia of brain activity.

56. The method of claim 50 wherein the step of assessing adaptability of the patient's perception of bodily self-image comprises presenting the patient with a sensory-perceptual paradox comprising stimulation of the visual sense, a visual illusion, visual effects, or digital imagery.

57. The method of claim 56 wherein the sensory paradox is at least partially created using a digital medium or a computer processor.

58. The method of claim 56 wherein the sensory-perceptual paradox involves at least one other sense, wherein the visual sense and other sense are each stimulated as part of the paradox.

59. The method of claim 58 wherein the visual and other sense are stimulated simultaneously or nearly simultaneously.

60. The method of claim 58 wherein the at least one other sense is somatosensory or tactile.

61. The method of claim 50 wherein the propensity for the patient to experience a response shift is a function of the time, duration, intensity and/or extent or any combination thereof, of the candidate's response to the assessment, or a function of the degree of paradox presented to the patient.

62. The method of claim 50 the patient's response to the assessment can be determined from an objective measure.

63. The method of claim 62 wherein the objective measure comprises a measure of neurological activity or brain activity, or an image thereof, or a measure of skin conductance.

64. The method of claim 62 wherein the objective measure is obtained via skin conductance resonance (SCR), electroencephalography (EEG), magnetic resonance imaging (MRI), computed tomography (CT), positron emission tomography (PET), electronystagmography (ENG), single photon emission computed tomography (SPECT) or by measuring evoked potentials.

65. The method of claim 60 wherein the sensory-perceptual paradox comprises stimulation of the visual sense by allowing the patient to view a simulated or artificial body or body part corresponding to an actual body or body part of the patient, and stimulation of the somatosensory sense by simultaneously touching the simulated or artificial body or body part and the patient's corresponding actual body or body part.

66. The method of claim 65 wherein the patient's corresponding actual body or body part is not in the patient's visual field while the simulated or artificial body or body part is visible to the patient.

67. The method of claim 65 wherein the degree of paradox is a function of how life-like the simulated or artificial body or body part is, wherein the more life-like the simulated body part is the lower the degree of paradox and the less life-like the simulated body part is, the higher the degree of paradox.

68. A method of selecting a course of therapy for a patient suffering from a terminal, chronic, progressive, or degenerative disease or condition, the method comprising the steps of:
determining which courses of therapy provide an option that might produce a desirable outcome for the patient;
for each option, considering the likelihood that the course of therapy will extend the life of the patient, alleviate the suffering of the patient, or otherwise improve the patient's physical or psychological situation;
assessing the likelihood that the patient will experience an improved psychological condition due to a response shift;
determining the cost-effectiveness for each option;
considering any other factors relevant to the therapy or patient; and
selecting a course of therapy for the patient based on the cost-effectiveness, and the likelihood that the patient will experience a response shift; and optionally, the other factors.

69. The method of claim 68 wherein the step of assessing the likelihood that the patient will experience an improved psychological condition due to a response shift comprises presenting the patient with a sensory-perceptual paradox comprising stimulation of the visual sense, a visual illusion, visual effects, or digital imagery, wherein the sensory paradox involves at least one other sense, wherein the visual sense and other sense are stimulated simultaneously or nearly simultaneously as part of the paradox.

70. The method of claim 60 wherein the at least one other sense is somatosensory or tactile.

71. The method of claim 69 wherein the likelihood that the patient will experience an improved psychological condition due to a response shift is a function of the time, duration, intensity and/or extent, or any combination thereof, of the patient's response to the assessment, or a function of the degree of paradox presented to the patient.

72. The method of claim 68 wherein the patient's response to the assessment can be determined from an objective measure comprising a measure of neurological activity or brain activity, or an image thereof, or a measure of skin conductance.

73. The method of claim 72 wherein the objective measure is obtained via skin conductance resonance (SCR), electroencephalography (EEG), magnetic resonance imaging (MRI), computed tomography (CT), positron emission tomography (PET), electronystagmography (ENG), single photon emission computed tomography (SPECT), magnetoencephalography (MEG), or superconducting quantum interference devices (SQUIDS), or by measuring evoked potentials.

74. The method of claim 69 wherein the sensory-perceptual paradox comprises stimulation of the visual sense by allowing the patient to view a simulated or artificial body or body part corresponding to an actual body or body part, and stimulation of the somatosensory sense by simultaneously touching the simulated or artificial body and the corresponding actual body or body part, wherein the corresponding actual body or body part is not in the patient's visual field while the simulated or artificial body or body part is visible to the patient.

75. The method of claim 71 wherein the degree of paradox is a function of how life-like the simulated or artificial body or body part is, wherein the more life-like the simulated body or body part is the lower the degree of paradox and the less life-like the simulated body or body part is, the higher the degree of paradox.

76. A method of conducting a quality of life (QOL) study comprising the steps of:
providing a plurality of subjects for the study;
for each subject:
providing over time a QOL assessment on each of a plurality, p, of occasions to obtain assessment data;
determining a score or scores for each such QOL assessment;
determining from the score or scores a baseline QOL response(s), based on the subject's score or scores for an initial number, n, of such occasions; such that p is much greater than n;
monitoring the subject's score or scores for each subsequent QOL assessment for unexpected deviations from the baseline QOL response; wherein an unexpected deviation is defined as part of the study;
ascertaining whether there are any known factors that may explain the unexpected deviation from the subject's baseline QOL response;
if there are no ascertainable factors that explain the unexpected deviation from the subject's baseline QOL response, assessing the propensity of a subject to experience an improved QOL due to a response shift; and
determining from the testing whether the subject shows a propensity to experience a response shift;
eliminating from the QOL study assessment data from subjects who are determined to show a propensity to experience an improved QOL due to a response shift;
completing the QOL study and analyzing the results thereof without the eliminated assessment data.

77. The method of claim 76 wherein the step of assessing the propensity of a subject to experience an improved psychological condition due to a response shift comprises presenting the subject with a sensory-perceptual paradox comprising stimulation of the visual sense, a visual illusion, visual effects, or digital imagery, wherein the sensory paradox involves at least one other sense, wherein the visual sense and other sense are stimulated simultaneously or nearly simultaneously as part of the paradox.

78. The method of claim 77 wherein the at least one other sense is somatosensory or tactile.

79. The method of claim 77 wherein the likelihood that the subject will experience an improved psychological condition due to a response shift is a function of the time, intensity, duration, or any combination thereof, of the subject's response to the assessment, or a function of the degree of paradox presented to the subject.

80. The method of claim 77 wherein the subject's response to the assessment can be determined from an objective measure comprising a measure of neurological activity or brain activity, or an image thereof, or a measure of skin conductance.

81. The method of claim 80 wherein the objective measure is obtained via skin conductance resonance (SCR), electroencephalography (EEG), magnetic resonance imaging (MRI), computed tomography (CT), positron emission tomography (PET), electronystagmography (ENG), single photon emission computed tomography (SPECT), magnetoencephalography (MEG), or superconducting quantum interference devices (SQUIDS), or by measuring evoked potentials.

82. The method of claim 78 wherein the sensory-perceptual paradox comprises stimulation of the visual sense by allowing the subject to view a simulated or artificial body or body part corresponding to an actual body part of the subject, and stimulation of the somatosensory sense by simultaneously touching the simulated or artificial body and the subject's corresponding actual body part, wherein the corresponding actual body part is not in the subject's visual field while the simulated or artificial body or body part is visible to the subject.

83. The method of claim 82 wherein the degree of paradox is a function of how life-like the simulated or artificial body or body part is, wherein the more life-like the simulated body part is the lower the degree of paradox and the less life-like the simulated body part is, the higher the degree of paradox.

84. A method for predicting that an individual is likely to be a placebo responder or is likely to experience a response shift, the method comprising the steps of
obtaining an objective measure of brain activity in the individual, and
determining therefrom whether the individual is likely to be a placebo responder or is likely to experience a response shift, wherein the measure of brain activity is correlated with performance in an assessment of adaptability of perception of bodily self-image.

85. The method of claim 84 wherein the objective measure is obtained via skin conductance resonance (SCR), electroencephalography (EEG), magnetic resonance imaging (MRI), computed tomography (CT), positron emission tomography (PET), electronystagmography (ENG), single photon emission computed tomography (SPECT), magnetoencephalography (MEG), or superconducting quantum interference devices (SQUIDS), or by measuring evoked potentials.

86. The method of claim 85 wherein the objective measure is obtained via functional MRI (fMRI), quantitative EEG (QEEG), magnetoencephalography (MEG), or superconducting quantum interference devices (SQUIDS).

87. A database comprising a collection of data useful for establishing a correlation between the objective measure of brain activity and an assessment of adaptability of perception of bodily self-image.
